(19) United States
(12) Patent Application Publication
(10) Pub. No.: US 2026/0265694 A1
(43) Pub. Date: Sep. 10, 2026

(54) USE OF A VITISIN COMPOUND FOR THE PRODUCTION OF HEMATOPOIETIC CELLS

(71) Applicants: ETABLISSEMENT FRANCAIS DU SANG, SAINT DENIS (FR); UNIVERSITE DE BORDEAUX, BORDEAUX (FR); INSTITUT NATIONAL DE LA SANTÉ ET DE LA RECHERCE MÉDICALE, PARIS (FR)

(72) Inventors: PHILIPPE BRUNET DE LA GRANGE, EYSINES (FR); MATHILDE HUART, BORDEAUX (FR); CHRISTELLE DEBEISSAT, LÉOGNAN (FR); MARYSE AVALON, SAINT AUBIN DE MÉDOC (FR); MARIJA VLASKI-LAFARGE, TALENCE (FR); ZORAN IVANOVIC, VILLENAVE D’ORNON (FR)

(21) Appl. No.: 18/997,107

(22) PCT Filed: Jul. 20, 2023
(86) PCT No.: PCT/EP2023/070191

§ 371 Date: Jan. 20, 2025

(30) Foreign Application Priority Data
Jul. 21, 2022 (EP) 22306094.8

(51) Int. Cl. C12N 5/078

(52) U.S. Cl. CPC C12N 5/0641; C12N 2500/35; C12N 2506/11

(57) ABSTRACT

The present invention relates to the use of a vitisin compound to amplify and/or differentiate hematopoietic cells. The invention also relates to a method for the in vitro production of erythrocyte progenitors comprising contacting hematopoietic cells with a cell culture medium comprising a vitisin compound.

### FIELD OF THE INVENTION

The present invention falls within the field of medicine. More particularly, it relates to novel processes for the production of erythrocyte progenitors and red blood cells.

### INTRODUCTION

Maintaining a constant supply of oxygen to tissues is essential for the survival of many living beings, in particular humans. Red blood cells transport oxygen throughout the body via the bloodstream. This transport is ensured by hemoglobin, a protein specific to red blood cells, which is capable of binding oxygen. When the red blood cells reach the tissues, the oxygen diffuses through the walls of the capillaries. The role of red blood cells is thus primordial.

Transfusion of red blood cells is necessary in emergency situations (hemorrhage) or pathological situations (blood diseases, cancers, etc.). In 2016, over 100 million blood bags were collected worldwide and distributed, so as to meet transfusion needs. To date, blood transfusions rely exclusively on blood from donors. However, since the health crisis linked to Covid-19, blood bag reserves have been dwindling, with donors no longer coming forward as regularly for blood donations.

In adults, red blood cell production or erythropoiesis takes place in the bone marrow, known as the hematopoietic marrow, which is present in flat bones and at the ends of long bones. In the bone marrow, multipotent stem cells, known as hematopoietic stem cells, differentiate successively into different types of erythrocyte progenitors, eventually forming mature red blood cells.

Media for the amplification and differentiation of hematopoietic cells are known to allow in vitro production of red blood cells. Various red blood cell production tests have been performed over time, notably as reviewed in Yoojin Seo, et al., Stem Cells International, vol. 2019, Article ID 9281329. In particular, different cell sources were tested and different protocols were developed, involving different cytokine cocktails, successions of steps suitable for the different stages of cell differentiation, modifying culture times (18 to 60 days) or using or not using a bioreactor.

The current processes present numerous difficulties for large-scale production, such as a low yield of red blood cells on conclusion of culturing, which does not allow efficient transfusion volumes to be achieved, and complex production systems, involving culturing in several successive and different steps, relatively long culture times to obtain mature red blood cells, an occasionally excessively rapid orientation toward maturation into red blood cells, or differentiation into cells that are incapable of performing the enucleation steps correctly (Akimov S. et al., 2005, Stem Cells, 23(9): 1423-1433; Hirose S. I. et al., 2013, Stem Cell Reports, 1(6): 499-508; Huang X., 2013, Mol. Ther., 22(2): 451-463). Finally, other processes resort to co-culturing with feeder cells (Kurita R. et al., 2013, PloS One, 8(3): e59890), which is both complex to implement and expensive.

It is thus seen that the development of novel, more efficient processes for producing red blood cells would make it possible to meet the needs for blood cell supplies.

The invention described herein is directed toward meeting these needs.

### SUMMARY OF THE INVENTION

According to a first aspect, the present invention relates to an in vitro process for producing erythrocytes, comprising:
a) providing multipotent hematopoietic cells, preferably of human origin, selected from the group consisting of hematopoietic stem cells (HSCs), multipotent progenitors (MPPs), common myeloid progenitors (CMPs), megakaryocyte-erythrocyte progenitors (MEPs), and combinations thereof, and optionally amplifying said multipotent hematopoietic cells;
b) inducing the differentiation of said multipotent hematopoietic cells into unipotent erythrocyte progenitors, and optionally amplifying said unipotent erythrocyte progenitors;
c) inducing the differentiation of said unipotent erythrocyte progenitors into erythrocyte precursors; and
d) inducing the maturation of the erythrocyte precursors into erythrocytes, and
e) optionally recovering the erythrocytes obtained;
wherein the cells are placed in contact with a vitisin compound in step a), in particular when it includes the cell amplification step, step b), step c) and/or step d).

The invention also relates to an in vitro process for producing unipotent erythrocyte progenitors, comprising:
a) providing multipotent hematopoietic cells, preferably of human origin, selected from the group consisting of hematopoietic stem cells (HSCs), multipotent progenitors (MPPs), common myeloid progenitors (CMPs), megakaryocyte-erythrocyte progenitors (MEPs), and combinations thereof, and optionally amplifying said multipotent hematopoietic cells; and
b) inducing the differentiation of said multipotent hematopoietic cells into unipotent erythrocyte progenitors, and optionally amplifying said unipotent erythrocyte progenitors; and
c) optionally, recovering the unipotent erythrocyte progenitors obtained;
wherein the cells are placed in contact with a vitisin compound in step a), in particular when it includes the cell amplification step and/or step b).

The invention also relates to an in vitro process for producing erythrocytes, comprising:
a) providing unipotent erythrocyte progenitors, preferably obtained via the process for producing unipotent erythrocyte progenitors according to the invention;
b) inducing the differentiation of said unipotent erythrocyte progenitors into unipotent erythrocyte precursors;
c) inducing the maturation of the erythrocyte precursors into erythrocytes, and
d) optionally recovering the erythrocytes obtained,
wherein the cells are placed in contact with a vitisin compound in step b) and/or step c).

In the processes according to the invention, the cells are placed in contact with the vitisin compound in a culture medium suitable for the nutritional requirements of hematopoietic cells, in particular a culture medium suitable for the amplification and/or differentiation and/or maturation of hematopoietic cells. Hematopoietic cells are preferentially selected from the group consisting of HSC, MPP, CMP, MEP cells, unipotent erythrocyte progenitors, erythrocyte precursors, and combinations thereof, preferably HSC or unipotent erythrocyte progenitors.

According to one embodiment, the cells are placed in contact with the vitisin compound i) during the amplification and/or differentiation of multipotent hematopoietic cells or ii) during the amplification and/or differentiation of unipotent erythrocyte progenitors.
According to another embodiment, the cells are placed in contact with the vitisin compound during the amplification and/or differentiation of multipotent hematopoietic cells, and during the amplification and/or differentiation of unipotent erythrocyte progenitors and optionally during the maturation of erythrocyte precursors.

In particular, the cells are placed in contact with a vitisin compound for at least 1 day, preferably at least 3, 5, 7, 10 or 12 days, during the amplification and/or differentiation of the multipotent hematopoietic cells, and/or for at least 1 day, preferably at least 3, 5, 7, 10 or 12 days, during the amplification and/or differentiation of the unipotent erythrocyte progenitors.

In the processes according to the invention, the vitisin compound, when placed in contact with the cells, is present in the culture medium at a concentration of between 1 μM and 500 μM, preferably between 5 μM and 100 μM.

According to a second aspect, the invention relates to a cell culture medium suitable for the amplification and/or differentiation and/or maturation of hematopoietic cells and comprising a vitisin compound.

In particular, the vitisin compound is present in the medium at a concentration of between 1 μM and 500 μM, preferably between 5 μM and 100 μM.

The invention also relates to the in vitro use of such a cell culture medium for (i) the amplification and/or differentiation of hematopoietic stem cells (HSCs), multipotent progenitors (MPPs), common myeloid progenitors (CMPs), megakaryocyte-erythrocyte progenitors (MEPs) or unipotent erythrocyte progenitors and/or (ii) for the differentiation and/or maturation of erythrocyte precursors, and/or (iii) the production of unipotent erythrocyte progenitors and/or erythrocytes.

According to a third aspect, the invention relates to the use of a vitisin compound to supplement a cell culture medium suitable for the amplification and/or differentiation and/or maturation of hematopoietic cells.

The invention also relates to the use of a vitisin compound to (i) stimulate the amplification and/or differentiation of hematopoietic stem cells (HSCs), multipotent progenitors (MPPs), common myeloid progenitors (CMPs), megakaryocyte-erythrocyte progenitors (MEPs) or unipotent erythrocyte progenitors and/or (ii) stimulate the differentiation and/or maturation of erythrocyte precursors, and/or (iii) stimulate the production of unipotent erythrocyte progenitors and/or erythrocytes.

In the processes, culture media and uses envisaged by the invention, the vitisin compound is chosen from the group consisting of vitisin B and derivatives thereof.

Preferably, the vitisin B derivative is a vitisin B isomer, preferably a vitisin B stereoisomer.

In particular, vitisin B derivatives have the formula:

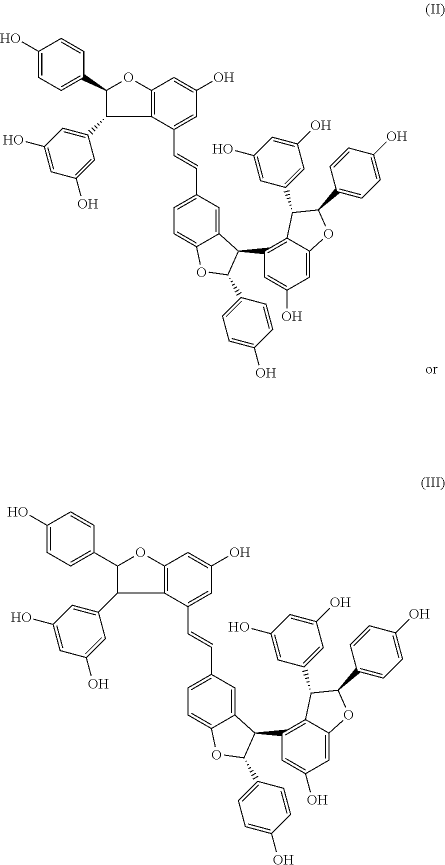 [SMILES: Oc1ccc([C@H]2Oc3cc(O)cc(/C=C/c4ccc5c(c4)[C@@H](c4cc(O)cc6c4[C@H](c4cc(O)cc(O)c4)[C@@H](c4ccc(O)cc4)O6)[C@H](c4ccc(O)cc4)O5)c3[C@@H]2c2cc(O)cc(O)c2)cc1 ; Oc1ccc(C2Oc3cc(O)cc(/C=C/c4ccc5c(c4)[C@@H](c4cc(O)cc6c4[C@H](c4cc(O)cc(O)c4)[C@@H](c4ccc(O)cc4)O6)[C@H](c4ccc(O)cc4)O5)c3C2c2cc(O)cc(O)c2)cc1]

Preferably, the vitisin compound is vitisin B.

### DETAILED DESCRIPTION OF THE INVENTION

The invention consists in using a vine-derived compound for the production of red blood cells. Vitisin B is a stilbene belonging to the polyphenol family, which may notably be extracted from vine shoots. Vitisin B has surprising advantages compared to other antioxidant molecules such as resveratrol.

The use of this molecule on human hematopoietic cells, in particular CD34⁺ cells, shows two important and reproducible effects:
1) vitisin B promotes the commitment of CD34⁺ multipotent hematopoietic cells to unipotent erythrocyte progenitors by increasing their amplification, and
2) vitisin B also makes it possible to accelerate the differentiation of progenitors into erythrocyte precursors and their enucleation, leading to mature cells: erythrocytes.
   The process according to the invention thus allows more efficient production of red blood cells.

The in vitro culture process according to the invention not only has the advantage of being simple and economical, but also paves the way for large-scale industrial production of erythrocyte progenitors and red blood cells. This makes it possible to reduce the risk of blood shortages or transfusion bottlenecks, while at the same time affording optimum safety for transfusion patients.

### Erythropoiesis and Hematopoietic Cells

Hematopoiesis is a continuous biological process that allows the renewal and maintenance of all blood cells: red blood cells (also referred to as erythrocytes), polymorphonuclear cells, platelets, monocytes and lymphocytes. Hematopoiesis is defined as the set of mechanisms which take place in the bone marrow and which, starting from hematopoietic stem cells (HSCs), ensure the continuous and regulated renewal of blood cells, i.e. red blood cells (RBCs, erythrocytes), white blood cells (WBCs, neutrophils, eosinophils, monocytes, lymphocytes) and platelets (Neildez-Nguyen et al., 2002).

Erythropoiesis is a biological process that allows the renewal of erythrocytes. During the erythropoiesis process, the hematopoietic stem cell (HSC) differentiates into multipotent progenitor (MPP), common myeloid progenitor (CMP), bipotent progenitor MEP (Megakaryocytic and Erythroid Progenitor), then into BFU-E (Burst Forming Unit—Erythroid) and CFU-E (Colony Forming Unit—Erythroid) progenitors, the latter two being irreversibly committed to the erythrocyte lineage (unipotent), and finally into erythrocyte precursors (Iwasaki & Akashi, Immunity. 2007 June; 26(6):726-40). The terminal differentiation phase of erythropoiesis allows the formation of the reticulocyte from erythrocyte precursors, after enucleation (expulsion of the nucleus), and then the formation of the erythrocyte.

The erythropoiesis process is well known to those skilled in the art and may be monitored by the expression kinetics of various specific markers, depending on the differentiation stage of the cells. In particular, it is possible to study the CD34 surface antigen to select a population of primitive CD34⁺ hematopoietic cells, the transferrin receptor (CD71), which is a marker of early erythrocyte differentiation, and also glycophorin A (GPA), which is a marker of terminal differentiation.

Glycophorin A (GPA) is a 10 kDa protein expressed at the membrane level of erythrocyte precursors (proerythroblasts) and erythrocytes. It is thus a marker of terminal erythrocyte differentiation, since it is absent from committed progenitors (BFU-E and CFU-E), is seen gradually on basophilic erythroblasts and remains stably expressed on erythrocytes.

The transferrin receptor 1 (CD71) is a 95 kDa protein expressed at the membrane of progenitors and erythrocyte precursors. Unlike GPA, CD71 expression reduces as differentiation proceeds, since erythrocytes do not express CD71. It is thus a marker of earlier erythrocyte differentiation.

The differentiation of erythrocyte cells is associated with the reduction and disappearance of CD71 and the acquisition of GPA, ultimately resulting in mature cells (erythrocytes and reticulocytes) characterized by the phenotype CD71⁻GPA⁺.

In particular, erythrocyte differentiation can be observed by flow cytometry, on the basis of specific expression markers such as membrane surface antigens (differentiation clusters (DCs)) such as the CD34, CD123, CD45RA, CD71 and GPA markers.

The various erythrocyte cells can also be observed under microscopy by means of May-Grünwald Giemsa (MGG) staining. Specifically, at each differentiation step, a reduction in cell and nucleus size is observed, ultimately leading to the formation of a mature anucleated erythrocyte cell. This cytology technique notably allows analysis of the maturation of erythrocyte precursors, and thus the detection and quantification of both the various precursors and mature erythrocytes.

The CFC (colony-forming cells) clonogenic assay also allows identification of the various erythrocyte cells. This assay quantifies the number of hematopoietic progenitors (HP) capable of forming colonies of mature erythrocyte cells when cultured in semi-solid media, such as media comprising methylcellulose. These media are preferably supplemented with the cytokines required for erythropoietic differentiation. These clonogenic assays allow progenitors with erythroid potential to be detected and quantified without the need for phenotypic markers.

These techniques are well known to those skilled in the art.

As used herein, the terms “hematopoietic cells”, “erythrocyte lineage cells” and “hematopoietic lineage cells” are equivalent and may be used interchangeably. These terms denote any one of the cells that allow an erythrocyte to be formed. These terms comprise multipotent hematopoietic cells, unipotent erythrocyte progenitors and erythrocyte precursors as defined below.

In particular, hematopoietic cells comprise HSCs, MPPs, CMPs, MEPs, BFU-Es, CFU-Es, proerythroblasts, basophilic erythroblasts, polychromatophilic erythroblasts and orthochromatic erythroblasts. The hematopoietic cells according to the invention are preferably obtained from a donor subject, in particular a healthy donor subject. In particular, hematopoietic cells, and HSCs in particular, may be obtained or prepared from a biological sample of a donor, such as a blood sample, e.g. peripheral blood, umbilical cord blood or placental blood, or a bone marrow sample.

Preferably, the cells as used in the present invention are cells of human origin.

As used herein, the term “multipotent hematopoietic cells” refers to multipotent CD34⁺ cells that are capable of differentiating to produce at least two cell types from among the various blood cells, namely white blood cells (immune cells), red blood cells and platelets. In the context of the present invention, the term “multipotent hematopoietic cells” comprises HSCs, MPPs, CMPs and MEPs. Preferably, MPPs have a phenotype CD34⁺LIN^{neg}CD123⁺CD45RA^{neg}, CMPs have a phenotype CD34⁺LIN^{neg}CD123⁺CD45RA^{neg} and MEPs have a phenotype CD34⁺LIN^{neg}CD123^{neg}CD45RA^{neg}. Preferably, the multipotent hematopoietic cells as used in the present invention are selected from the group consisting of HSCs, MPPs, CMPs and MEPs, and combinations thereof. More particularly preferably, the multipotent hematopoietic cells as used in the present invention are HSCs, optionally in combination with MPPs, CMPs and/or MEPs.

As used herein, the term “hematopoietic stem cell” or “HSC” refers to multipotent stem cells that are capable of differentiating to produce all the blood cells, namely white blood cells (immune cells), red blood cells and platelets. According to preferred embodiments, the HSCs are human HSCs. HSCs bear particular markers, such as high expression of the primitive marker CD34, absence of expression of the marker CD45RA (isoform of phosphotyrosine phosphatase CD45), and optionally expression of the marker CD133. Preferably, HSCs also express the IL3 receptor (CD123). The term “HSC” notably includes the cells known as “Side Population” (SP) cells. The term “SP cells” combines all cells in which it is possible to demonstrate, by flow cytometry, a capacity for efflux of a vital dye such as Hoescht or Dye Cycle Violet (DCV). Preferably, SP cells thus have a phenotype CD34⁺DCV^{low}.

Multipotent hematopoietic cells may be obtained from a variety of sources, according to processes that are well known to those skilled in the art. They may notably be isolated from bone marrow, cytapheresis, whole blood or even umbilical cord blood (or placental blood), for example using an immunomagnetic system or a sorting system based on the presence of specific membrane receptors (for example CD133, CD123, CD45 and/or CD34).

Multipotent hematopoietic cells may also be obtained by differentiating embryonic stem cells or induced pluripotent stem (iPS) cells, preferably induced pluripotent stem cells. Techniques for differentiating pluripotent stem cells into multipotent hematopoietic cells are well known to those skilled in the art. Several protocols have been published, notably the protocol of Lengerke C. et al. (2009, Ann. N.Y. Acad. Sci., 1176:219-27) consisting of a 17-day differentiation via an intermediate step of embryoid bodies and by virtue of the combination of the following cytokines: SCF, Flt-3 ligand, IL-3, IL-6, G-CSF and BMP-4.

As used herein, the term “embryonic stem cell” refers to cells derived from the internal cell mass of the blastocyst, and which have the capacity of leading to the formation of all the body's tissues (mesoderm, endoderm, ectoderm), including germ-line cells. The pluripotency of embryonic stem cells may be evaluated by the presence of markers such as the transcription factors OCT4 and NANOG, and surface markers such as SSEA3/4, Tra-1-60 and Tra-1-81. Embryonic stem cells may be obtained without destroying the embryo from which they are derived, for example via a technique described by Chung et al. (Cell Stem Cell, 2008, 2(2): 113-117). In a particular embodiment, and for legal or ethical reasons, the embryonic stem cells are non-human embryonic stem cells. In another particular embodiment, the embryonic stem cells used in the invention are human embryonic stem cells, preferably obtained without destroying the embryo from which they are derived. The embryos used are preferably supernumerary embryos obtained in the context of a parental project after obtaining the regulatory and ethical authorizations in accordance with the laws in force.

As used herein, the term “induced pluripotent stem cell” (iPSC, IPSc or iPS), refers to pluripotent stem cells obtained by genetic reprogramming of differentiated somatic cells, and having a morphology and potential for self-renewal and pluripotency partly similar to those of embryonic stem cells. These cells are notably positive for pluripotency markers, notably alkaline phosphatase staining and expression of NANOG, SOX2, OCT4 and SSEA3/4 proteins. Processes for producing induced pluripotent stem cells are well known to those skilled in the art and are notably described in the articles by Yu et al. (Science, 2007, 318 (5858): 1917-1920), Takahashi et al. (Cell, 2007, 131(5): 861-872) and Nakagawa et al. (Nat. Biotechnol., 2008, 26(1): 101-106).

As used herein, the term “unipotent erythrocyte progenitors” refers to progenitor cells obtained by differentiation of HSCs during erythropoiesis, and which have the ability to divide and subsequently differentiate into erythrocyte precursors and then into red blood cells by enucleation. In particular, after the MEP stage (last multipotent stage), cells gradually acquire markers specific to erythrocyte progenitors. First, the CD71 marker (transferrin receptor) is seen, followed by the appearance of Glycophorin A (GPA or CD235a). Unipotent erythrocyte progenitors include BFU-E (Burst Forming Unit—E) and CFU-E (Colony Forming Unit—E) cells, which are irreversibly committed to the erythrocyte lineage. BFU-E (Burst Forming Unit—E) cells have a phenotype CD34⁺CD36^{neg}GPA^{neg}CD123^{neg}CD71^{low}. CFU-E (Colony Forming Unit—E) cells have a phenotype CD34^{neg}CD36^{neg}GPA^{neg}CD123^{neg}CD71^{high}.

As used herein, the term “erythrocyte precursor” defines cells obtained by differentiation of unipotent erythrocyte progenitors and that are capable of producing reticulocytes. The term encompasses proerythroblasts, basophilic erythroblasts, polychromatophilic erythroblasts and orthochromatic erythroblasts. The distinction between these cells is possible on a cytological basis by staining the cells with May-Grünwald Giemsa (MGG) staining, analyzing the coloration and the nucleus/cytoplasm ratio, via techniques well known to those skilled in the art.

As used herein, the terms “red blood cell”, “mature red blood cell”, “red corpuscle”, “erythrocyte” and “mature erythrocyte” are equivalent and may be used interchangeably. The term “erythrocyte” refers to an enucleated cell having characteristic markers of erythrocyte maturation. Erythrocytes notably express glycophorin A (CD235a), but do not express the CD36 marker. Their identification may be based on cytological criteria well known to those skilled in the art, such as cell size and the absence of a cell nucleus.

According to one embodiment, the multipotent hematopoietic cells used in the processes according to the invention to produce unipotent erythrocyte progenitors or to produce erythrocytes are obtained by sampling from a donor or are derived from cells obtained from a donor. In particular, the erythrocyte progenitors or erythrocytes thus obtained may be intended for transplantation into a recipient patient, notably by transfusion.

Donors and recipients may be the same or different individuals. In one embodiment, the donor and the recipient are the same individual. In another preferred embodiment, the donor is different from the recipient. In this case, the donor is preferably a healthy donor, in particular an individual free of hematological pathologies.

### Vitisin Compound

The invention consists in using a vitisin compound in the ex vivo or in vitro production of multipotent hematopoietic cells and/or unipotent erythrocyte progenitors and/or red blood cells, in particular to promote the amplification and/or differentiation of hematopoietic cells. In particular, the vitisin compound allows faster and greater erythrocyte differentiation of multipotent hematopoietic cells, in particular HSCs, into unipotent erythrocyte progenitors. Furthermore, it allows faster and greater erythrocyte differentiation of unipotent erythrocyte progenitors into erythrocyte precursors and/or erythrocyte precursors into red blood cells.

As used herein, the term “vitisin compound” refers to a stilbene belonging to the polyphenol family, which may be extracted from vine shoots, for example. The term denotes vitisin B or a derivative thereof.
Preferably, the term “vitisin compound” does not include compounds chosen from resveratrol, pallidol, ε-viniferin, ampelopsin, oxyresveratrol, piceatannol and/or gnetol.
According to preferred embodiments, the vitisin compound is vitisin B.
Vitisin B is a tetramer of resveratrol present in plants of the genus Vitis, such as Vitis vinifera.
Vitisin B is also known under the names
1,3-Benzenediol, 5-[(2R,2'S,3R,3'S)-5-[(1E)-2-[(2S,3S)-3-(3,5-dihydroxyphenyl)-2,3-dihydro-6-hydroxy-2-(4-hydroxyphenyl)-4-benzofuranyl]ethenyl]-2,2′,3,3′-tetrahydro-6′-hydroxy-2,2′-bis(4-hydroxyphenyl)[3,4′-bibenzofuran]-3′-yl]-(9CI, ACI);
1,3-Benzenediol, 5-[5-[2-[3-(3,5-dihydroxyphenyl)-2,3-dihydro-6-hydroxy-2-(4-hydroxyphenyl)-4-benzofuranyl]ethenyl]-2,2′,3,3′-tetrahydro-6′-hydroxy-2,2′-bis(4-hydroxyphenyl)[3,4′-bibenzofuran]-3′-yl]-, [2R-[2α,3β(2'S*,3'S*),5[E(2S*,3S*)]]]-(ZCI); or
5-[(2R,2'S,3R,3'S)-5-[(1E)-2-[(2S,3S)-3-(3,5-Dihydroxyphenyl)-2,3-dihydro-6-hydroxy-2-(4-hydroxyphenyl)-4-benzofuranyl]ethenyl]-2,2′,3,3′-tetrahydro-6′-hydroxy-2,2′-bis(4-hydroxyphenyl)[3,4′-bibenzofuran]-3′-yl]-1,3-benzenediol (ACI), and in particular has the CAS No. 142449-90-9.

In particular, the vitisin B as used according to the invention has the following formula (I):

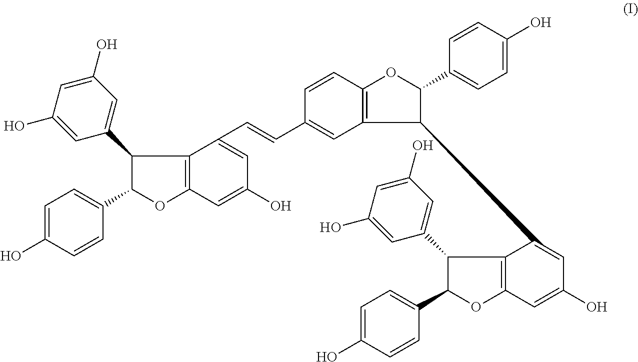 [SMILES: Oc1ccc([C@H]2Oc3ccc(/C=C/c4cc(O)cc5c4[C@H](c4cc(O)cc(O)c4)[C@@H](c4ccc(O)cc4)O5)cc3[C@@H]2c2cc(O)cc3c2[C@@H](c2cc(O)cc(O)c2)[C@H](c2ccc(O)cc2)O3)cc1]

Vitisin B may notably be in the form of a salt or hydrate.

According to other embodiments, the vitisin compound is a vitisin B derivative. Preferably, the vitisin derivative is a vitisin B isomer, preferably a vitisin B stereoisomer. In particular, vitisin B stereoisomers include R or S stereoisomers at any one of the vitisin B stereochemical sites.

Examples of isomers may be, but are not limited to, a compound having a CAS No. chosen from CAS 165883-77-2, CAS180580-73-8, CAS142507-86-6, CAS 1373138-12-5, CAS1807631-15-7, CAS879897-47-9, CAS879897-46-8 and CAS681004-56-8; or a compound of formulae (I), (II) and (III) described below and combinations thereof.

In particular, the vitisin B derivative may be a vitisin B stereoisomer, for example as described in patent application WO 2015/126129. Preferably, the vitisin B derivative is a stereoisomer which depends on the double bond, in the Z position (compound 20 of patent application WO 2015/126129) or in the E position (compound 22 of patent application WO 2015/126129).

The vitisin B derivative may notably have the following formula (II) or (III):

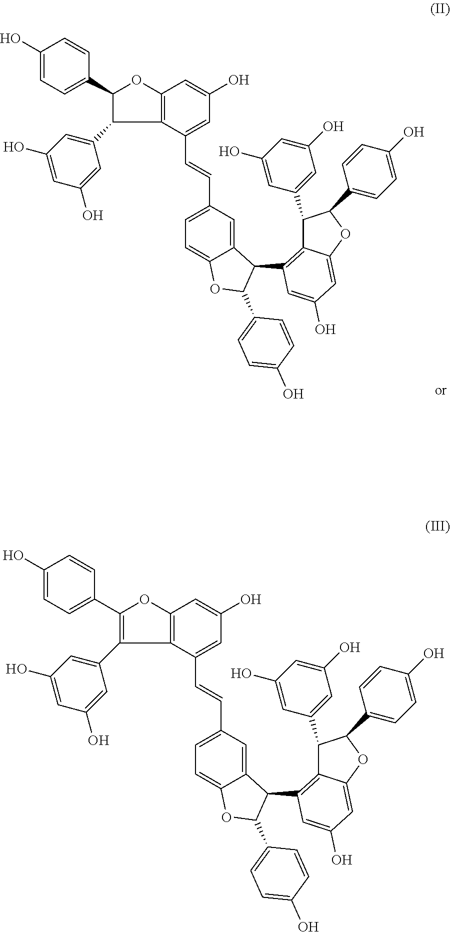

According to certain particular embodiments, the vitisin compound is chosen from the group consisting of compounds of formulae (I), (II) and (III), and combinations thereof.

Thus, according to certain embodiments, the vitisin compound may be chosen from vitisin B and a vitisin B derivative having a CAS number chosen from CAS142449-90-9, CAS 165883-77-2, CAS180580-73-8, CAS142507-86-6, CAS 1373138-12-5, CAS1807631-15-7, CAS879897-47-9, CAS879897-46-8 and CAS681004-56-8 or a compound of formulae (I), (II) and (III) described above and combinations thereof.

Placing the vitisin compound in contact with the cells may be performed by adding the vitisin compound to a culture medium, in particular a medium suitable for growing hematopoietic cells. The culture medium is preferably suitable for the nutritional requirements of hematopoietic cells and in particular suitable for the growth and/or differentiation of hematopoietic cells, into which the vitisin compound is added. Such media are known to a person skilled in the art and some are more specifically described below.

Preferably, the vitisin compound is used at a concentration of between about 0.5 μM and about 1 mM, about 0.5 μM and about 500 μM, about 0.5 μM and about 200 μM, about 0.5 μM and about 100 μM, between about 0.5 μM and about 50 μM, between about 0.5 μM and about 40 μM, between about 0.5 μM and about 30 μM, between about 0.5 μM and about 20 μM, between about 0.5 μM and about 15 μM or between about 0.5 μM and about 10 μM, about 5 μM and about 1 mM, about 5 μM and about 500 μM, about 5 μM and about 200 μM, about 5 μM and about 100 μM, between about 5 μM and about 75 μM, between about 5 μM and about 50 μM, between about 5 M and about 40 μM, between about 5 μM and about 30 μM, between about 5 μM and about 20 μM, between about 5 μM and about 15 μM or between about 5 μM and about 10 μM, preferably between about 10 μM and about 1 mM, about 10 μM and about 500 μM, about 10 μM and about 200 μM, about 10 μM and about 100 μM, 10 μM and about 75 μM, between about 10 μM and about 50 μM, between about 10 μM and about 40 μM, between about 10 μM and about 30 μM, between about 10 μM and about 20 μM, preferably at a concentration of about 10 μM. Preferably, the vitisin compound is used at a concentration of between 1 μM and 500 μM, preferentially between 5 M and 100 IM. In particular, vitisin-B is used at a concentration of about 0.5, 1, 5, 10, 15, 20, 25, 30, 35, 40, 45, 50, 55, 60, 65, 70, 75, 80, 85, 80, 90, 95 or 100 μM.

### Production Processes

The present invention relates to processes for the production or preparation of cells of the erythrocyte lineage.

In particular, the production processes according to the invention may comprise:
a) the amplification, differentiation and/or maturation of cells of the erythrocyte lineage, and
b) optionally, the recovery of multiplied and/or differentiated and/or mature erythrocyte lineage cells, and
c) optionally, storage of obtained/recovered erythrocyte lineage cells.

Preferably, the erythrocyte lineage cells are chosen from HSCs, MPPs, CMPs, MEPs, BFU-Es, CFU-Es, and erythrocyte precursors, namely proerythroblasts, basophilic erythroblasts, polychromatophilic erythroblasts, orthochromatic erythroblasts, reticulocytes and/or erythrocytes, and any combinations thereof.

In the processes according to the invention, the vitisin compound may be placed in contact, preferably in a suitable culture medium, with cells of the erythrocyte lineage during cell amplification, differentiation and/or maturation, preferably during cell amplification and/or differentiation.

In certain embodiments, the vitisin compound is placed in contact with multipotent hematopoietic cells, in particular cells chosen from HSC, MPP, CMP and MEP cells, and combinations thereof, so as to amplify said cells and/or to obtain unipotent erythrocyte progenitors, erythrocyte precursors and/or erythrocytes by differentiation of said cells.

In other embodiments, the vitisin compound is placed in contact with unipotent erythrocyte progenitors in order to amplify said progenitors and/or to obtain erythrocyte precursors and/or erythrocytes by differentiation of said progenitors.

In other embodiments, the vitisin compound is placed in contact with erythrocyte precursors so as to obtain erythrocytes by differentiation and maturation of said precursors.

In other embodiments, the vitisin compound is placed in contact with CD34⁺ cells, i.e. cells chosen from HSC, MPP, CMP, MEP and BFU-E cells, and combinations thereof, in order to amplify said cells and/or to obtain CFU-E progenitors, erythrocyte precursors and/or erythrocytes by differentiation of said cells.

In particular, the present invention relates to an in vitro process for producing erythrocytes, comprising:
a) providing multipotent hematopoietic cells, preferably of human origin, selected from the group consisting of hematopoietic stem cells (HSCs), multipotent progenitors (MPPs), common myeloid progenitors (CMPs), megakaryocyte-erythrocyte progenitors (MEPs), and combinations thereof, and optionally amplifying said multipotent hematopoietic cells;
b) inducing the differentiation of said multipotent hematopoietic cells into unipotent erythrocyte progenitors, and optionally amplifying said unipotent erythrocyte progenitors;
c) inducing the differentiation of said unipotent erythrocyte progenitors into erythrocyte precursors; and
d) inducing the maturation of the erythrocyte precursors into erythrocytes, and optionally recovering the erythrocytes obtained.

In this process, cells (multipotent hematopoietic cells, unipotent erythrocyte progenitors and/or erythrocyte precursors) are placed in contact with a vitisin compound in step a) (when this step comprises the amplification of said multipotent hematopoietic cells), step b), step c) and/or step d).

Preferably, the present invention relates to an in vitro process for producing erythrocytes, comprising:
a) providing multipotent hematopoietic cells, preferably of human origin, selected from the group consisting of hematopoietic stem cells (HSCs), multipotent progenitors (MPPs), common myeloid progenitors (CMPs), megakaryocyte-erythrocyte progenitors (MEPs), and combinations thereof, and amplifying said multipotent hematopoietic cells;
b) inducing the differentiation of said multipotent hematopoietic cells into unipotent erythrocyte progenitors, and optionally amplifying said unipotent erythrocyte progenitors;
c) inducing the differentiation of said unipotent erythrocyte progenitors into erythrocyte precursors; and
d) inducing the maturation of the erythrocyte precursors into erythrocytes, and
optionally recovering the erythrocytes obtained.

In this process, the cells (multipotent hematopoietic cells, unipotent erythrocyte progenitors and/or erythrocyte precursors) are placed in contact with a vitisin compound in step a), step b), step c) and/or step d).

The present invention also relates to an in vitro process for producing unipotent erythrocyte progenitors, comprising:
a) providing multipotent hematopoietic cells, preferably of human origin, selected from the group consisting of hematopoietic stem cells (HSCs), multipotent progenitors (MPPs), common myeloid progenitors (CMPs), megakaryocyte-erythrocyte progenitors (MEPs), and combinations thereof, and optionally amplifying said multipotent hematopoietic cells; and
b) inducing the differentiation of said multipotent hematopoietic cells into unipotent erythrocyte progenitors, and optionally amplifying said unipotent erythrocyte progenitors; and
c) optionally, recovering the unipotent erythrocyte progenitors obtained.

In this process, the cells (multipotent hematopoietic cells and/or unipotent erythrocyte progenitors) are placed in contact with a vitisin compound in step a) (when this step comprises the amplification of said multipotent hematopoietic cells) and/or step b).

Preferably, the present invention also relates to an in vitro process for producing unipotent erythrocyte progenitors, comprising:
a) providing multipotent hematopoietic cells, preferably of human origin, selected from the group consisting of hematopoietic stem cells (HSCs), multipotent progenitors (MPPs), common myeloid progenitors (CMPs), megakaryocyte-erythrocyte progenitors (MEPs), and combinations thereof, and amplifying said multipotent hematopoietic cells; and
b) inducing the differentiation of said multipotent hematopoietic cells into unipotent erythrocyte progenitors, and optionally amplifying said unipotent erythrocyte progenitors; and
c) optionally, recovering the unipotent erythrocyte progenitors obtained.

In this process, the cells (multipotent hematopoietic cells and/or unipotent erythrocyte progenitors) are placed in contact with a vitisin compound in step a) and/or step b).

This process notably aims to induce the differentiation of multipotent hematopoietic cells into unipotent erythrocyte progenitors, and to allow either the amplification of this progenitor population while at the same time retaining their ability to differentiate into red blood cells at a later stage, or to continue the differentiation of this progenitor population into erythrocyte precursors and red blood cells.

The present invention also relates to an in vitro process for producing erythrocyte precursors, comprising:
a) providing unipotent erythrocyte progenitors;
b) inducing the differentiation of said unipotent erythrocyte progenitors into erythrocyte precursors; and
optionally recovering the erythrocyte precursors obtained.

In this process, the cells (unipotent erythrocyte progenitors and/or erythrocyte precursors) are placed in contact with a vitisin compound in step b).

Preferably, the unipotent erythrocyte progenitors provided in step a) are obtained via the process for the production of unipotent erythrocyte progenitors according to the invention.

The present invention also relates to an in vitro process for producing erythrocytes, comprising:
a) providing unipotent erythrocyte progenitors;
b) inducing the differentiation of said unipotent erythrocyte progenitors into erythrocyte precursors;
c) inducing the maturation of the erythrocyte precursors into erythrocytes, and optionally recovering the erythrocytes obtained.

In this process, the cells (unipotent erythrocyte progenitors and/or erythrocyte precursors) are placed in contact with a vitisin compound in step b) and/or step c).

Preferably, the unipotent erythrocyte progenitors provided in step a) are obtained via the process for producing unipotent erythrocyte progenitors according to the invention.

The present invention also relates to an in vitro process for producing erythrocytes, comprising:
a) providing erythrocyte precursors; and
b) inducing the maturation of the erythrocyte precursors into erythrocytes, and
optionally recovering the erythrocytes obtained.

In this process, erythrocyte precursor cells are placed in contact with a vitisin compound in step b).

Preferably, the erythrocyte precursors provided in step a) are obtained via a process for producing erythrocyte precursors according to the invention.

The multipotent hematopoietic cells provided in the processes according to the invention may be obtained via any method known to those skilled in the art. As indicated above, they may notably be isolated from bone marrow, cytapheresis, whole blood, umbilical cord or placental blood samples, for example using an immunomagnetic system or a sorting system based on the presence of specific membrane receptors. They may also be obtained by differentiation of embryonic stem cells or induced pluripotent stem (iPS) cells as indicated above. Optionally, prior to their use in one of the processes according to the invention, these multipotent hematopoietic cells may be amplified by culturing in a medium that is suitable for the amplification of these cells. The multipotent hematopoietic cells provided comprise HSCs, MPPs, CMPs and/or MEPs, preferably HSCs and optionally MPPs, CMPs and/or MEPs. The multipotent hematopoietic cells provided may also comprise any other cell types. In particular, these cells may be CD34⁺ cells and may also comprise BFU-Es.

The unipotent erythrocyte progenitors provided in the processes according to the invention may be obtained via the process for producing unipotent erythrocyte progenitors according to the invention, or via any other method known to those skilled in the art. In particular, these progenitors may be isolated from bone marrow, cytapheresis, whole blood, umbilical cord or placental blood samples, for example using an immunomagnetic system or a sorting system based on the presence of specific membrane receptors. They may also be obtained by differentiation of multipotent hematopoietic cells using any method known to those skilled in the art. Optionally, prior to their use in one of the processes according to the invention, these unipotent erythrocyte progenitors may be amplified by culturing in a medium that is suitable for the amplification of these cells. The unipotent erythrocyte progenitors provided comprise BFU-Es and/or CFU-Es. They may also comprise other cell types such as HSCs, MPPs, CMPs and/or MEPs.

The erythrocyte precursors provided in the processes according to the invention may be obtained via a process for producing erythrocyte precursors according to the invention, or via any other method known to those skilled in the art. In particular, these erythrocyte precursors may be isolated from bone marrow, cytapheresis, whole blood, umbilical cord or placental blood samples, for example using an immunomagnetic system or a sorting system based on the presence of specific membrane receptors. They may also be obtained by differentiation of multipotent hematopoietic cells or unipotent erythrocyte progenitors using any method known to those skilled in the art. Optionally, prior to their use in one of the processes according to the invention, these erythrocyte precursors may be amplified by culturing in a medium that is suitable for the amplification of these cells.

In the processes according to the invention, the cells can be placed in contact with the vitisin compound in a culture medium suitable for the nutritional requirements of hematopoietic cells. According to the steps during which this placing in contact is performed, this culture medium may be a culture medium suitable for the amplification and/or differentiation and/or maturation of hematopoietic cells.

The culture medium suitable for the nutritional requirements of hematopoietic cells may be any medium known to those skilled in the art for meeting the needs of these cells, and more particularly for meeting the needs of cells selected from the group consisting of HSC, MPP, CMP, MEP cells, unipotent erythrocyte progenitors, erythrocyte precursors, and combinations thereof. This medium satisfies the minimum survival requirements of the cells to be cultured and allows, depending on the situation and the supplements provided, the maintenance, amplification, differentiation and/or maturation of hematopoietic cells. Numerous culture media suitable for the nutritional requirements of the hematopoietic cells of interest are known to those skilled in the art and are commercially available, such as Iscove's modified Dulbecco's medium (IMDM medium), the Stem Span SFEM II medium (Stemcell technologies) or the “StemMACS HSC Expansion Media XF, Human” (Miltenyi Biotec). The composition of these media is well known.

Preferably, in the processes according to the invention, the vitisin compound is placed in contact with the cells in a medium that is suitable for the amplification and/or differentiation and/or maturation of hematopoietic cells. Such media are well known to a person skilled in the art. Examples of such media are notably mentioned in the “Culture medium” section below. In general, media suitable for cell amplification, differentiation and maturation may be obtained from the same basic medium suitable for the nutritional requirements of hematopoietic cells, for example the IMDM medium or the Stem Span SFEM II medium, and differ essentially in their cytokine composition.

As used herein, the term “growth”, “multiplication” or “amplification” refers to the multiplication of cells. Thus, a medium that is suitable for the amplification of hematopoietic cells is one that allows the multiplication of multipotent hematopoietic cells, namely HSCs, MPPs, CMPs and/or MEPs, unipotent erythrocyte progenitors and/or erythrocyte precursors. A culture medium suitable for the amplification of a cell type may also be suitable for the differentiation of this cell type. Conversely, when only amplification is desired, for example to increase the pool of multipotent hematopoietic cells or unipotent erythrocyte progenitors, the culture medium suitable for amplification stimulates little or no cell differentiation.

In the processes according to the invention, multipotent hematopoietic cells may be amplified prior to differentiation. This amplification may take place in any medium suitable for the amplification of multipotent hematopoietic cells, notably HSCs, and known to those skilled in the art. In particular, multipotent hematopoietic cells may be amplified in a medium suitable for the nutritional requirements of hematopoietic cells, for example the IMDM medium or the Stem Span SFEM II medium, comprising, or supplemented with, G-SCF, SCF, FLT3-L and TPO, in particular in the concentrations described below in the “Culture medium” section.

In the processes according to the invention, the erythrocyte progenitors may be amplified prior to differentiation. This amplification may be performed in any medium that is suitable for the amplification of erythrocyte progenitors and known to those skilled in the art. In particular, erythrocyte progenitors can be amplified in a medium suitable for the nutritional requirements of hematopoietic cells, for example the IMDM medium or the Stem Span SFEM II medium, comprising, or supplemented with, G-SCF, SCF, FLT3-L and TPO, in particular in the concentrations described below in the “Culture medium” section.

In the processes according to the invention, erythrocyte precursors may be amplified prior to maturation. This amplification may take place in any medium that is suitable for the amplification of erythrocyte precursors and known to those skilled in the art. In particular, erythrocyte precursors may be amplified in a medium suitable for the nutritional requirements of hematopoietic cells, for example the IMDM medium or the Stem Span SFEM II medium, comprising, or supplemented with, EPO, hydrocortisone, suitable growth factors, transferrin, serum and/or plasma, or any combination thereof, in particular in the concentrations described below in the “Culture medium” section.

The term “differentiation” refers to the acquisition by cultured cells of features that were not present in the cells initially used to inoculate the medium. In the present case, this term refers to the acquisition of features of cells that are more advanced in the erythropoiesis process. Thus, a medium that is suitable for the differentiation of hematopoietic cells is therefore a medium that allows the differentiation of multipotent hematopoietic cells, such as HSCs, MPPs, CMPs and/or MEPs, into unipotent erythrocyte progenitors and/or the differentiation of unipotent erythrocyte progenitors into erythrocyte precursors and/or the differentiation of erythrocyte precursors so as to obtain, after maturation, erythrocytes. Typically, a culture medium that is suitable for the differentiation of a cell type is also suitable for cell amplification.

In the processes according to the invention, multipotent hematopoietic cells may be differentiated into unipotent erythrocyte progenitors. This differentiation may take place in any medium that is suitable for differentiating multipotent hematopoietic cells, notably HSCs, and known to those skilled in the art. In particular, multipotent hematopoietic cells may be differentiated in a medium suitable for the nutritional requirements of hematopoietic cells, for example the IMDM medium or the Stem Span SFEM II medium, comprising IL3, IL6, SCF, serum albumin, in particular bovine albumin, insulin, transferrin and optionally EPO.

In the processes according to the invention, erythrocyte progenitors can be differentiated into erythrocyte precursors. This differentiation may take place in any medium that is suitable for the differentiation of erythrocyte progenitors and known to those skilled in the art. In particular, erythrocyte progenitors may be differentiated in a medium suitable for the nutritional requirements of hematopoietic cells, for example the IMDM medium or the Stem Span SFEM II medium, comprising IL3, SCF, hydrocortisone and optionally EPO.

As used herein, the term “maturation” refers to the process by which erythrocyte precursors become red blood cells, notably involving cell enucleation. Thus, a medium that is suitable for hematopoietic cell maturation is therefore one that allows erythrocyte precursors to mature into erythrocytes. Typically, a medium that is suitable for the amplification and/or differentiation of hematopoietic cells does not promote this maturation. The maturation of erythrocyte precursors is notably reflected by the expression of erythrocyte maturation markers such as CD235a, and by enucleation.

In the processes according to the invention, erythrocyte precursors may be matured to obtain erythrocytes. This maturation may take place in any medium that is suitable for the maturation of erythrocyte precursors and known to those skilled in the art. In particular, erythrocyte precursors can be matured in a medium suitable for the nutritional requirements of hematopoietic cells, for example the IMDM medium or the Stem Span SFEM II medium, comprising, or supplemented with, EPO, preferably 2 or 3 IU/ml EPO. Preferably, the medium does not comprise any cytokines. Preferably, erythrocyte precursor maturation is induced at a high cell concentration, for example above 5 000 000 cells/ml of culture.

Thus, in the processes according to the invention,
the amplification of multipotent hematopoietic cells may be performed by placing the multipotent hematopoietic cells in a culture medium suitable for the amplification of multipotent hematopoietic cells, in particular a medium as described above or in the “Culture medium” section, typically for a period of 3 to 15 days, preferably 5 to 8 days;
the amplification of erythrocyte progenitors may be performed by placing the erythrocyte progenitors in a culture medium suitable for the amplification of erythrocyte progenitors, in particular a medium as described above or in the “Culture medium” section, typically for a period of 3 to 15 days, preferably 4 to 12 days;
the amplification of erythrocyte precursors may be performed by placing the erythrocyte precursors in a culture medium suitable for the amplification of erythrocyte precursors, in particular a medium as described above or in the “Culture medium” section, typically for a period of 2 to 6 days, preferably 2 to 4 days;
the induction of the differentiation of multipotent hematopoietic cells into erythrocyte progenitors may be performed by placing the multipotent hematopoietic cells in a culture medium suitable for differentiation of multipotent hematopoietic cells, in particular a medium as described above or in the “Culture medium” section, typically for a period of 3 to 10 days, preferably 3 to 6 days;
the induction of the differentiation of erythrocyte progenitors into erythrocyte precursors may be performed by placing the erythrocyte progenitors in a culture medium that is suitable for the differentiation of erythrocyte progenitors, in particular a medium as described above or in the “Culture medium” section, typically for a period of 3 to 10 days, preferably 3 to 6 days;
the induction of erythrocyte precursor maturation may be performed by placing the erythrocyte precursors in a culture medium suitable for erythrocyte precursor maturation, in particular a medium as described above or in the “Culture medium” section, typically for a period of 5 to 15 days, preferably 8 to 12 days.

Techniques for modifying the composition of a culture medium are well known to those skilled in the art. In particular, adding a molecule or increasing its concentration may be done directly in the pre-existing culture medium, and removing a molecule or reducing its concentration may be done by centrifuging the cells and resuspending in a new culture medium or by diluting the culture medium.

The times given above for each step of the process are for guidance only. These times may be readily defined/adjusted by a person skilled in the art by observing the number and/or nature of the cells in culture.

In the processes according to the invention, the cells may be placed in contact with the vitisin compound during
the step of amplifying multipotent hematopoietic cells, and/or
the step of differentiating multipotent hematopoietic cells into unipotent erythrocyte progenitors, and/or
the step of amplifying unipotent erythrocyte progenitors, and/or
the step of differentiating unipotent erythrocyte progenitors into erythrocyte precursors; and/or
the step of amplifying erythrocyte precursors, and/or
the step of inducing maturation of erythrocyte precursors into erythrocytes.

According to certain particular embodiments, the cells are placed in contact with the vitisin compound during each of these steps.

According to other particular embodiments, the cells are placed in contact with the vitisin compound during the step of amplifying multipotent hematopoietic cells and/or the step of differentiating multipotent hematopoietic cells into unipotent erythrocyte progenitors.

According to other particular embodiments, the cells are placed in contact with the vitisin compound during the step of amplifying unipotent erythrocyte progenitors and/or the step of differentiating unipotent erythrocyte progenitors into erythrocyte precursors.

According to other particular embodiments, the cells are placed in contact with the vitisin compound during the step of amplifying unipotent erythrocyte progenitors and/or the step of differentiating unipotent erythrocyte progenitors into erythrocyte precursors and/or the step of differentiating unipotent erythrocyte progenitors into erythrocyte precursors.

According to other particular embodiments, the cells are placed in contact with the vitisin compound during (i) the step of amplifying multipotent hematopoietic cells and/or the step of differentiating multipotent hematopoietic cells into unipotent erythrocyte progenitors and (ii) the step of amplifying unipotent erythrocyte progenitors and/or the step of differentiating unipotent erythrocyte progenitors into erythrocyte precursors.

According to other particular embodiments, the cells are placed in contact with the vitisin compound during (i) the step of amplifying multipotent hematopoietic cells and/or the step of differentiating multipotent hematopoietic cells into unipotent erythrocyte progenitors, and (ii) the step of amplifying unipotent erythrocyte progenitors and/or the step of differentiating unipotent erythrocyte progenitors into erythrocyte precursors and (iii) the step of maturing erythrocyte precursors into erythrocytes.

According to other particular embodiments, the cells are placed in contact with the vitisin compound during the step of differentiating multipotent hematopoietic cells into unipotent erythrocyte progenitors, and the step of differentiating unipotent erythrocyte progenitors into erythrocyte precursors; and optionally during the step of amplifying multipotent hematopoietic cells and/or the step of amplifying unipotent erythrocyte progenitors.

According to other particular embodiments, the cells are placed in contact with the vitisin compound during the step of differentiating multipotent hematopoietic cells into unipotent erythrocyte progenitors, and the step of differentiating unipotent erythrocyte progenitors into erythrocyte precursors; and optionally during the step of amplifying multipotent hematopoietic cells and/or the step of amplifying unipotent erythrocyte progenitors, and optionally during the step of maturing erythrocyte precursors into erythrocytes.

Preferably, the cells are placed in contact with the vitisin compound at least during the step of amplifying unipotent erythrocyte progenitors and/or the step of differentiating unipotent erythrocyte progenitors into erythrocyte precursors.

When the cells are placed in contact with the vitisin compound during a step of the process, the vitisin compound may be present in the culture medium throughout the step or during part of this step. Preferably, the vitisin compound is present throughout the step. Alternatively, the vitisin compound may be present for at least 1 day, preferably at least 3, 5, 7, 10 or 12 days during the step.

In particular, the cells may be placed in contact with a vitisin compound for at least 1 day, preferably for at least 3, 5, 7, 10 or 12 days, during the amplification and/or differentiation of multipotent hematopoietic cells, and/or for at least 1 day, preferably for at least 3, 5, 7, 10 or 12 days, during the amplification and/or differentiation of unipotent erythrocyte progenitors.

Preferably, the cells are placed in contact with a vitisin compound for at least 1 day, preferably for at least 3, 5, 7, 10 or 12 days, during the amplification of multipotent hematopoietic cells, for at least 1 day, preferably for at least 3, 5, 7, 10 or 12 days, during differentiation of multipotent hematopoietic cells, for at least 1 day, preferably for at least 3, 5, 7, 10 or 12 days, during amplification of unipotent erythrocyte progenitors and for at least 1 day, preferably for at least 3, 5, 7, 10 or 12 days, during differentiation of unipotent erythrocyte progenitors.

In particular, the cells may be placed in contact with a vitisin compound for between 2 and 15 days, in particular between 3 and 12 days, particularly between 3 and 10 days.

According to particular embodiments, the processes according to the invention may comprise
the amplification of multipotent hematopoietic cells in a culture medium suitable for the amplification of multipotent hematopoietic cells, preferably an IMDM medium supplemented with G-SCF, SCF, FLT3-L and TPO, and also comprising a vitisin compound, preferably vitisin B, typically for a period of 3 to 15 days, preferably 5 to 8 days;
and
induction of the differentiation of multipotent hematopoietic cells into erythrocyte progenitors in a culture medium suitable for the differentiation of multipotent hematopoietic cells, preferably an IMDM medium supplemented with IL3, IL6, SCF, serum albumin, in particular bovine albumin, insulin, transferrin and optionally EPO, and also comprising a vitisin compound, preferably vitisin B, typically for a period of 3 to 10 days, preferably 3 to 6 days.

According to particular embodiments, the processes according to the invention may comprise
the amplification of erythrocyte progenitors in a culture medium suitable for the amplification of erythrocyte progenitors, preferably an IMDM or Stem Span SFEM II medium supplemented with G-SCF, SCF, FLT3-L and TPO, and also comprising a vitisin compound, preferably vitisin B, typically for a period of 3 to 15 days, preferably 4 to 12 days; and
induction of the differentiation of erythrocyte progenitors into erythrocyte precursors in a culture medium that is suitable for the differentiation of erythrocyte progenitors, preferably an IMDM or Stem Span SFEM II medium supplemented with IL3, SCF, hydrocortisone and optionally EPO, and also comprising a vitisin compound, preferably vitisin B, typically for a period of 3 to 10 days, preferably 3 to 6 days.

According to particular embodiments, the processes according to the invention may comprise the induction of the maturation of erythrocyte precursors into erythrocytes in a culture medium suitable for the maturation of erythrocyte precursors, preferably an EPO-supplemented IMDM or Stem Span SFEM 11 medium, preferably comprising 2 or 3 IU/ml EPO, and also comprising a vitisin compound, preferably vitisin B, typically for a period of 5 to 15 days, preferably 8 to 12 days. Preferably, this maturation medium does not comprise any cytokines.

According to certain embodiments, hematopoietic cells are cultured in a culture medium suitable for the nutritional requirements of hematopoietic cells for less than 50 days, less than 45 days, less than 40 days, less than 35 days or less than 30 days. In particular, hematopoietic cells are cultured in a culture medium suitable for the nutritional requirements of hematopoietic cells for between 3 and 35 days, between 7 and 35 days, between 15 and 35 days, between 25 and 35 days, between 28 and 35 days, between 7 and 28 days, between 15 and 28 days, between 20 and 28 days, preferably for about 28 or 35 days.
In a particular embodiment, when the process comprises the production of erythrocytes from unipotent progenitors, hematopoietic cells are cultured in a culture medium suitable for the nutritional requirements of hematopoietic cells for 25 to 30 days, in particular about 28 days.
In a particular embodiment, when the process comprises the production of erythrocytes from multipotent hematopoietic cells, in particular from HSCs, the hematopoietic cells are cultured in a culture medium suitable for the nutritional requirements of the hematopoietic cells for 30 to 40 days, in particular about 35 days.

In the processes according to the invention, the cell culture steps, namely the amplification, differentiation and/or maturation steps, are preferably performed at a temperature suitable for the multiplication and/or differentiation of hematopoietic cells. Preferably, the temperature is about 37° C.

The CO₂ and/or O₂ content is preferably controlled during the cell culture steps. Preferably, the CO₂ content is between 2% and 10%, between 3% and 7% or between 4% and 6%, and is preferentially about 5%. The O₂ content is preferably between 1% and 30%, between 5% and 25%, between 10% and 25% or between 15% and 25%, and is preferentially about 20%.

In a particularly preferred manner, the cells are cultured at about 37° C., with a CO₂ content of 5% and/or an O₂ content of 20%.

Preferably, the hematopoietic cells of interest in the present invention are cultured at a concentration of between 200 and 10 000 cells/ml, preferably between 500 and 2000 cells/ml, and even more preferably at about 1000 cells/ml.

Preferably, during the culture steps, hematopoietic cells are placed in contact with mesenchymal cells (MSCs) or stromal cells. The term “stromal cells”, “MSCs” or “mesenchymal cells” denotes non-hematopoietic bone marrow cells, such as endothelial cells, non-striated vascular cells (fibrotic cells), adipocytes and macrophages. Mesenchymal stem cells are present in various tissues of the adult body, mainly in bone marrow but also in adipose tissue. They may differentiate into numerous cell types, including osteoblasts, chondrocytes, myocytes and adipocytes, and thus produce and/or repair bone and fat tissue. Within the medullary niche, mesenchymal cells and hematopoietic cells are in contact. In particular, multipotent hematopoietic cells and hematopoietic progenitors are notably extrinsically regulated within medullary hematopoietic niches, involving intercellular contacts and diffusible factors. Mesenchymal stem cells secrete growth factors that control the proliferation and differentiation of these hematopoietic cells.

Preferably, stromal or mesenchymal cells are grown under suitable culture conditions so that they adhere or do not adhere to a substrate surface.

Hematopoietic cells may be cocultured with mesenchymal cells (MSCs) to form a confluent cell mat. A person skilled in the art may use any known technique to obtain a confluent MSC cell mat.

According to certain particular embodiments, in the processes according to the invention, the step of differentiating multipotent hematopoietic cells into unipotent erythrocyte progenitors, and/or the step of differentiating unipotent erythrocyte progenitors into erythrocyte precursors, and/or the step of maturing erythrocyte precursors, are performed in coculture with MSC cells. Preferably, the steps of differentiating multipotent hematopoietic cells into unipotent erythrocyte progenitors and of differentiating unipotent erythrocyte progenitors into erythrocyte precursors, and optionally the step of maturation of erythrocyte precursors, are performed in coculture with MSC cells.

When the processes according to the invention comprise a step of co-culturing with MSCs, the culture medium may also comprise fetal calf serum (FCS), in particular at a concentration of between about 1% and about 10%, preferably at a concentration of between about 1% and about 5%.

The processes according to the invention may also comprise one or more additional steps.

The processes according to the invention may also notably comprise a step of collecting blood from an individual, preferably umbilical cord blood, placental blood and/or peripheral blood.

The processes according to the invention may also comprise one or more cell sorting steps, in particular a cell selection step based on the expression of markers such as CD34, CD36, CD123, CD235a, CD117, CD71 and/or GPA, or any combination thereof. This preliminary step may in particular be performed via an immunomagnetic technique using antibodies directed against various phenotypic markers of the cells of interest, in particular as described in the “Erythropoiesis and hematopoietic cells” section.

The processes for producing erythrocytes according to the invention may also comprise a nucleated cell removal step. This step notably allows a homogeneous population comprising only mature erythrocytes to be obtained.

The production processes according to the invention may also comprise a step of recovering the cells obtained. This step may be performed via any technique known to those skilled in the art, notably filtration, centrifugation and removal of the culture medium.

Production processes according to the invention may also comprise a step of washing the obtained/recovered cells. This step may be performed via any technique known to those skilled in the art, notably a succession of filtration, centrifugation and resuspension steps.

The production processes according to the invention may also comprise a step of storing the obtained/recovered cells. This step may be performed via any technique known to those skilled in the art, notably freezing. In particular, the recovered cells may be mixed with a volume-to-volume solution of decomplemented fetal calf serum (FCS) and cryoprotectant prior to freezing.

In the processes according to the invention, the vitisin compound, preferably vitisin-B, may be added to any of the culture media suitable for the amplification, differentiation and/or maturation steps.

In particular, the vitisin compound, preferably vitisin B, may be present in the medium at a concentration of between about 0.5 μM and about 1 mM, about 0.5 μM and about 500 μM, about 0.5 μM and about 200 μM, about 0.5 μM and about 100 μM, between about 0.5 μM and about 50 μM, between about 0.5 μM and about 40 μM, between about 0.5 μM and about 30 μM, between about 0.5 μM and about 20 μM, between about 0.5 μM and about 15 μM or between about 0.5 μM and about 10 μM, about 5 μM and about 1 mM, about 5 μM and about 500 μM, about 5 μM and about 200 μM, about 5 μM and about 100 μM, between about 5 μM and about 75 μM, between about 5 M and about 50 μM, between about 5 μM and about 40 μM, between about 5 μM and about 30 μM, between about 5 μM and about 20 μM, between about 5 μM and about 15 μM or between about 5 μM and about 10 μM, preferably between about 10 μM and about 1 mM, about 10 μM and about 500 μM, about 10 μM and about 200 μM, about 10 μM and about 100 μM, 10 μM and about 75 μM, between about 10 μM and about 50 μM, between about 10 μM and about 40 μM, between about 10 μM and about 30 μM, between about 10 μM and about 20 μM, preferably at a concentration of about 10 μM. Preferably, the vitisin compound is used at a concentration of between 1 μM and 500 μM, preferentially between 5 μM and 100 μM. In particular, vitisin-B may be used at a concentration of about 0.5, 1, 5, 10, 15, 20, 25, 30, 35, 40, 45, 50, 55, 60, 65, 70, 75, 80, 85, 80, 90, 95 or 100 μM.

### Culture Medium

The present invention also relates to a cell culture medium comprising a vitisin compound, in particular a cell culture medium suitable for the nutritional requirements of hematopoietic cells, in particular multipotent, pluripotent or unipotent hematopoietic cells, preferably selected from the group consisting of HSC, MPP, CMP, MEP cells, unipotent erythrocyte progenitors, erythrocyte precursors, and combinations thereof, and comprising a vitisin compound. In particular, the cell culture medium according to the invention may be a medium suitable for the maintenance, amplification, differentiation and/or maturation of hematopoietic cells, preferably cells selected from the group consisting of HSC, MPP, CMP, MEP cells, unipotent erythrocyte progenitors, erythrocyte precursors, and combinations thereof. This medium may notably be used in the various steps of the processes according to the invention.

The culture medium according to the invention may be any basic medium known to those skilled in the art to be suitable for the nutritional requirements of hematopoietic cells, in particular multipotent hematopoietic cells, unipotent progenitors and/or erythrocyte precursors, to which a vitisin compound is added. Numerous culture media suitable for the nutritional requirements of hematopoietic cells are known to those skilled in the art and are commercially available, such as Iscove's modified Dulbecco's medium (IMDM medium), the Stem Span SFEM II medium (Stemcell technologies) or else the “StemMACS HSC Expansion Media XF, Human” (Miltenyi Biotec).

The cell culture medium is preferably chosen from an Iscove's modified Dulbecco's medium (IMDM medium) and a Stem Span SFEM II medium (Stemcell technologies). Preferably, the culture medium is an Iscove's modified Dulbecco culture medium (IMDM_PAN BIOTECH). The IMDM culture medium is notably obtained from Dulbecco's Modified Eagle's Medium (DMEM), enriched not only with sodium selenite, but also with vitamins and amino acids. The compositions of IMDM and Stem Span SFEM II media are well known in the literature. The IMDM medium notably comprises amino acids, vitamins, salts, D-glucose, HEPES and sodium pyruvate.

The culture medium according to the invention may be liquid, semisolid or solid. A person skilled in the art knows how to solidify a culture medium, notably by adding agar.

The culture medium according to the invention may comprise one or more amino acids, antibiotics, vitamins, salts, minerals or lipids. The culture medium according to the invention may also comprise other compounds conducive to the amplification and/or differentiation of hematopoietic cells, notably growth or survival factors, for instance as described in patent application WO 2010/096746. In particular, the cell culture medium may be supplemented with erythropoietin (EPO), Stem Cell Factor (SCF), hydrocortisone (HC), growth factors, interleukin 3 (IL3), interleukin 6 (IL6), interleukin 11 (IL11), transferrin, heparin, insulin, thrombopoietin, FMS-type tyrosine kinase 3 ligand (Flt3-L), granulocyte colony-stimulating factor (G-CSF), L-glutamine, inositol, an antibiotic such as penicillin and/or streptomycin, folic acid, mono-thioglycerol, iron nitrate, iron sulfate, BIT 9500 Supplement (Stem Cell Technologies), serum albumin, serum and/or plasma, or any combinations thereof. The concentrations of these various compounds are readily determined by a person skilled in the art on the basis of supplier recommendations or general knowledge of the field. A person skilled in the art knows how to adjust the presence of such compounds suitable for hematopoietic cell culture.

The culture medium according to the invention may optionally comprise an antibiotic, for example penicillin and/or streptomycin, at a concentration of between about 0.1% and about 10%, preferably between about 0.5% and about 5%, more preferably at a concentration of about 1%.

The compounds in the culture medium according to the invention are preferably human compounds obtained via recombinant or purification techniques.

Preferably, the culture medium according to the invention notably comprises:
IL-3, preferably human IL-3, at a concentration of between about 1 ng/mL and about 20 ng/mL, preferably between about 5 ng/mL and about 15 ng/mL, and more preferably at a concentration of about 5 or 10 ng/mL; and/or
IL-6, preferably human IL-6, at a concentration of between about 1 ng/mL and about 20 ng/mL, preferably between about 5 ng/mL and about 15 ng/mL, more preferably at a concentration of about 10 ng/mL; and/or
SCF at a concentration of between about 1 ng/mL and about 500 ng/mL, preferably between about 10 ng/mL and about 200 ng/mL, preferentially between about 50 ng/mL and about 150 ng/mL, preferably between about 50 ng/mL and about 100 ng/mL, most preferably about 100 ng/mL; and/or
EPO, preferably human EPO, at a concentration of between about 0.5 IU/mL and about 10 IU/mL, preferably between about 1 IU/mL and about 5 IU/mL, and more preferably at a concentration of about 2 or 3 IU/mL; and/or
FLT3-L at a concentration of between about 1 ng/mL and about 500 ng/mL, preferably between about 10 ng/mL and about 200 ng/mL, preferentially between about 50 ng/mL and about 150 ng/mL, preferentially a concentration of 100 ng/mL; and/or
TPO at a concentration of between about 0.1 ng/mL and about 200 ng/mL, preferably between about 1 ng/mL and about 100 ng/mL, preferentially between about 10 ng/mL and about 50 ng/mL, preferentially a concentration of 20 ng/mL; and/or
G-CSF at a concentration of between about 0.1 ng/mL and about 200 ng/mL, preferably between about 1 ng/mL and about 100 ng/mL, preferentially between about 5 ng/mL and about 25 ng/mL, preferentially a concentration of 10 ng/mL, and/or
transferrin, preferably human transferrin, at a concentration of between about 50 μg/mL and about 400 μg/mL, preferably between about 50 μg/mL and about 200 μg/mL, preferably between 100 μg/mL and 150 μg/mL, more preferably at a concentration of about 120 μg/mL; and/or
insulin, preferably human insulin, at a concentration of between about 1 μg/mL and about 50 μg/mL, preferably between about 5 μg/mL and about 20 μg/mL, more preferably at a concentration of about 10 μg/mL; and/or
heparin, preferably human heparin, at a concentration of between about 0.5 U/mL and about 10 U/mL, preferably between about 1 U/mL and about 5 U/mL, more preferably at a concentration of about 3 U/mL; and/or
L-glutamine at a concentration of between about 0.1 mM and about 50 mM, preferably between about 1 mM and about 15 mM, preferably between about 1 mM and about 5 mM, most preferably about 4 mM; and/or
inositol at a concentration of between about 10 μg/mL and about 200 μg/mL, preferably between about 20 μg/mL and about 100 μg/mL, preferably between 20 μg/mL and 60 μg/mL, more preferably at a concentration of about 40 μg/mL; and/or
folic acid at a concentration of between about 0.1 μg/mL and about 100 μg/mL, preferably between about 1 μg/mL and about 100 μg/mL, preferably between 5 μg/mL and 20 μg/mL, more preferably at a concentration of about 10 μg/mL; and/or
mono-thioglycerol at a concentration of between about 10⁻⁵ M and about 10⁻³ M, preferably between about 0.1×10⁻⁴ M and about 10×10⁻⁴ M, preferably between 10⁻⁴ M and about 5×10⁻⁴ M, more preferably at a concentration of about 1.6×10⁻⁴ M; and/or
hydrocortisone (HC) at a concentration of between about 10⁻⁷ μM and about 10⁻⁸ M, preferably at a concentration of about 10⁻⁶ M; and/or
fetal calf serum at a concentration of between about 1% and about 10%, preferably between about 3% and about 7%, more preferably at a concentration of about 5%; and/or
iron nitrate at a concentration of between about 1 ng/mL and about 200 ng/mL, preferably between about 10 ng/mL and about 150 ng/mL, preferentially between about 50 ng/mL and about 150 ng/mL, more preferably at a concentration of about 90 ng/mL;
iron sulfate at a concentration of between about 100 ng/mL and about 1500 ng/mL, preferably between about 500 ng/mL and about 1500 ng/mL, preferentially between about 800 ng/mL and about 1000 ng/mL, and more preferably at a concentration of about 900 ng/mL;
human or bovine serum albumin at a concentration of between about 1 mg/mL and about 50 mg/mL, preferably between about 5 mg/mL and about 20 mg/mL, more preferably at a concentration of about 10 mg/mL;
albumin at a concentration of between about 1% and about 10%, preferably between about 3% and about 7%, and even more preferably at a concentration of about 4%, and/or
BIT 9500 supplement (Stem Cell Technologies) at a concentration of between about 1% and about 30%, preferably between about 10% and about 20%, more preferably at a concentration of about 15%; and/or;
serum, plasma or serum pool, preferably of human origin, at a concentration of between about 1% and about 10%, preferably between about 3% and about 7%, more preferably at a concentration of about 5%, and/or platelet lyzate, preferably of human origin, at a concentration of between about 0.05% and about 0.5%, preferably between about 0.1% and about 0.5%, more preferably at a concentration of about 0.3%;
or any combination thereof.

According to certain embodiments, the culture medium according to the invention is a culture medium comprising:
L-glutamine at a concentration of between about 0.1 mM and about 50 mM, preferably between about 1 mM and about 15 mM, preferably between about 1 mM and about 5 mM, most preferably about 4 mM; and/or
inositol at a concentration of between about 10 μg/mL and about 200 μg/mL, preferably between about 20 μg/mL and about 100 μg/mL, preferably between 20 μg/mL and 60 μg/mL, more preferably at a concentration of about 40 μg/mL; and/or
folic acid at a concentration of between about 0.1 μg/mL and about 100 μg/mL, preferably between about 1 μg/mL and about 100 μg/mL, preferably between 5 μg/mL and g/mL, more preferably at a concentration of about 10 μg/mL; and/or
mono-thioglycerol at a concentration of between about 10⁻⁵ M and about 10⁻³ M, preferably between about 0.1×10⁻⁴ M and about 10×10⁻⁴ M, preferably between 10⁻⁴ M and about 5×10⁻⁴ M, more preferably at a concentration of about 1.6×10⁻⁴ M; and/or
transferrin, preferably human transferrin, at a concentration of between about 50 μg/mL and about 400 μg/mL, preferably between about 50 μg/mL and about 200 μg/mL, preferably between 100 μg/mL and 150 μg/mL, more preferably at a concentration of about 120 μg/mL; and/or
insulin, preferably human insulin, at a concentration of between about 1 μg/mL and about 50 μg/mL, preferably between about 5 μg/mL and about 20 μg/mL, more preferably at a concentration of about 10 μg/mL; and/or
iron nitrate at a concentration of between about 1 ng/mL and about 200 ng/mL, preferably between about 10 ng/mL and about 150 ng/mL, preferentially between about 50 ng/mL and about 150 ng/mL, more preferably at a concentration of about 90 ng/mL;
iron sulfate at a concentration of between about 100 ng/mL and about 1500 ng/mL, preferably between about 500 ng/mL and about 1500 ng/mL, preferentially between about 800 ng/mL and about 1000 ng/mL, more preferably at a concentration of about 900 ng/mL;
human serum albumin at a concentration of between about 1 mg/mL and about 50 mg/mL, preferably between about 5 mg/mL and about 20 mg/mL, more preferably at a concentration of about 10 mg/mL; and/or
IL-3, preferably human IL-3, at a concentration of between about 1 ng/mL and about 20 ng/mL, preferably between about 5 ng/mL and about 15 ng/mL, more preferably at a concentration of about 5 ng/mL; and/or
SCF at a concentration of between about 1 ng/mL and about 500 ng/mL, preferably between about 10 ng/mL and about 200 ng/mL, preferably between about 50 ng/mL and about 150 ng/mL, preferably between about 50 ng/mL and about 100 ng/mL, most preferably about 100 ng/mL; and/or
EPO, preferably human EPO, at a concentration of between about 0.5 IU/mL and about 10 IU/mL, preferably between about 1 IU/mL and about 5 IU/mL, more preferably at a concentration of about 3 IU/mL; and/or
hydrocortisone (HC) at a concentration of between about 10⁻⁷ M and about 10⁻⁸ M, preferably at a concentration of about 10⁻⁶ M.

According to certain embodiments, the culture medium is an amplification medium, notably suitable for the amplification of multipotent hematopoietic cells or unipotent erythrocyte progenitors, for example an IMDM or Stem Span II medium, also comprising:
G-CSF at a concentration of between about 0.1 ng/mL and about 200 ng/mL, preferably between about 1 ng/mL and about 100 ng/mL, preferentially between about 5 ng/mL and about 25 ng/mL, preferentially a concentration of 10 ng/mL, and
SCF at a concentration of between about 1 ng/mL and about 500 ng/mL, preferably between about 10 ng/mL and about 200 ng/mL, preferentially between about 50 ng/mL and about 150 ng/mL, preferentially a concentration of 100 ng/mL, and
FLT3-L at a concentration of between about 1 ng/mL and about 500 ng/mL, preferably between about 10 ng/mL and about 200 ng/mL, preferentially between about 50 ng/mL and about 150 ng/mL, preferentially a concentration of 100 ng/mL, and
TPO at a concentration of between about 0.1 ng/mL and about 200 ng/mL, preferably between about 1 ng/mL and about 100 ng/mL, preferentially between about 10 ng/mL and about 50 ng/mL, preferentially a concentration of 20 ng/mL.

According to certain embodiments, the culture medium is an amplification medium, notably suitable for the amplification of erythrocyte precursors, for example an IMDM or Stem Span II medium, also comprising:
SCF in particular at a concentration of between about 1 ng/mL and about 500 ng/mL, preferably between about 10 ng/mL and about 200 ng/mL, preferentially between about 50 ng/mL and about 150 ng/mL, preferably between about 50 ng/mL and about 100 ng/mL, most preferably about 100 ng/mL;
hydrocortisone (HC) in particular at a concentration of between about 10⁻⁷ M and about 10⁻⁸ M, preferably at a concentration of about 10⁻⁶ M and/or optionally,
transferrin (BIT), preferably human transferrin, at a concentration of between about 50 μg/mL and about 400 μg/mL, preferably between about 50 μg/mL and about 200 μg/mL, preferably between 100 μg/mL and 150 μg/mL, more preferably at a concentration of about 120 μg/mL;
EPO, preferably human EPO, at a concentration of between about 0.5 IU/mL and about 10 IU/mL, preferably between about 1 IU/mL and about 5 IU/mL, more preferably at a concentration of about 2 IU/mL, and/or
serum, plasma or serum pool, preferably of human origin, at a concentration of between about 1% and about 10%, preferably between about 3% and about 7%, more preferably at a concentration of about 5%, and/or platelet lysate, preferably of human origin, at a concentration of between about 0.05% and about 0.5%, preferably between about 0.1% and about 0.5%, more preferably at a concentration of about 0.3%.

According to certain other embodiments, the culture medium is a differentiation medium, notably enabling the differentiation of multipotent hematopoietic cells into unipotent erythrocyte progenitors, for example an IMDM or Stem Span II medium, also comprising:
IL-3, preferably human IL-3, at a concentration of between about 1 ng/mL and about 20 ng/mL, preferably between about 5 ng/mL and about 15 ng/mL, more preferably at a concentration of about 10 ng/mL;
IL-6, preferably human IL-6, at a concentration of between about 1 ng/mL and about 20 ng/mL, preferably between about 5 ng/mL and about 15 ng/mL, more preferably at a concentration of about 10 ng/mL;
SCF at a concentration of between about 1 ng/mL and about 500 ng/mL, preferably between about 10 ng/mL and about 200 ng/mL, preferentially between about 25 ng/mL and about 100 ng/mL, preferentially a concentration of about 50 ng/mL,
bovine serum albumin (BSA) at a concentration of between about 1 mg/mL and about 50 mg/mL, preferably between about 5 mg/mL and about 20 mg/mL, more preferably at a concentration of about 10 mg/mL;
transferrin (BIT), preferably human transferrin, at a concentration of between about 50 μg/mL and about 400 μg/mL, preferably between about 50 μg/mL and about 200 μg/mL, preferably between 100 μg/mL and 150 μg/mL, more preferably at a concentration of about 120 μg/mL;
insulin, preferably human insulin, at a concentration of between about 1 μg/mL and about 50 μg/mL, preferably between about 5 μg/mL and about 20 μg/mL, more preferably at a concentration of about 10 μg/mL;
optionally EPO, preferably human EPO, at a concentration of between about 0.5 IU/mL and about 10 IU/mL, preferably between about 1 IU/mL and about 5 IU/mL, more preferably at a concentration of about 2 IU/mL, and
optionally fetal calf serum (FCS), in particular at a concentration of between about 1% and about 10%, preferably at a concentration of between about 1% and about 5%.

BSA, insulin and transferrin may notably be provided by the use of BIT 9500 (Stem Cell Technologies) at a concentration of between about 1% and about 30%, preferably between about 10% and about 20%, and more preferably at a concentration of about 15%.

According to certain embodiments, the culture medium is a differentiation medium, notably suitable for the differentiation of unipotent progenitors into erythrocyte precursors, for example an IMDM or Stem Span II medium, also comprising:
IL-3, preferably human IL-3, in particular at a concentration of between about 1 ng/mL and about 20 ng/mL, preferably between about 5 ng/mL and about 15 ng/mL, more preferably at a concentration of about 5 ng/mL; and
SCF in particular at a concentration of between about 1 ng/mL and about 500 ng/mL, preferably between about 10 ng/mL and about 200 ng/mL, preferentially between about 50 ng/mL and about 150 ng/mL, preferably between about 50 ng/mL and about 100 ng/mL, most preferably about 100 ng/mL; and
hydrocortisone (HC) in particular at a concentration of between about 10⁻⁷ M and about 10⁻⁸ M, preferably at a concentration of about 10⁻⁶ M and/or optionally, and
optionally, EPO, preferably human EPO, in particular at a concentration of between about 0.5 IU/mL and about 10 IU/mL, preferably between about 1 IU/mL and about 5 IU/mL, more preferably at a concentration of about 3 IU/mL; and
optionally, fetal calf serum (FCS), in particular at a concentration of between about 1% and about 10%, preferably at a concentration of between about 1% and about 5%.

According to certain other embodiments, the culture medium is a maturation medium; for example, an IMDM or Stem Span II medium not comprising cytokines. Preferably, the maturation medium does not comprise any cytokines, but is supplemented with erythropoietin (about 2 or 3 IU/mL).

According to a very particular embodiment, the culture medium according to the invention comprises a vitisin compound and the compounds of Table 1, preferably in the concentrations mentioned in Table 1.

TABLE 1
Example of a culture medium composition suitable for hematopoietic cell culture.

| | Concentration | | Concentration |
|---|---|---|---|
| Compounds | (mM) | Compounds | (mM) |
| Glycine | 0.4 | Choline chloride | 0.03 |
| L-Alanine | 0.28 | D-Calcium pantothenate | 0.008 |
| L-Alanyl-L-glutamine | 4.0 | Folic acid | 0.009 |
| L-Arginine hydrochloride | 0.4 | Niacinamide | 0.033 |
| L-Asparagine | 0.19 | Pyridoxal hydrochloride | 0.02 |
| L-Aspartic acid | 0.23 | Riboflavin | 0.001 |
| L-Cystine | 0.22 | Thiamine hydrochloride | 0.012 |
| L-Glutamic acid | 0.51 | Vitamin B12 | 9.59E−6 |
| L-Histidine hydrochloride-H₂O | 0.2 | i-Inositol | 0.04 |
| L-Isoleucine | 0.80 | CaCl₂-2H₂O | 1.97 |
| L-Leucine | 0.80 | MgSO₄-7H₂O | 1.67 |
| L-Lysine hydrochloride | 0.80 | KCl | 4.4 |
| L-Methionine | 0.20 | KNO₃ | 7.52E−4 |
| L-Phenylalanine | 0.4 | NaHCO₃ | 36.0 |
| L-Proline | 0.35 | NaCl | 77.59 |
| L-Serine | 0.4 | NaH₂PO₄-2H₂O | 1.02 |
| L-Threonine | 0.80 | Na₂SeO₃-5H₂O | 6.46E−5 |
| L-Tryptophan | 0.08 | Dextrose | 25.0 |
| L-Tyrosine disodium | 0.46 | HEPES | 25.03 |
| L-Valine | 0.80 | Phenol Red | 0.04 |
| Biotin | 5.33E−5 | Sodium pyruvate | 1.0 |

According to another embodiment, the culture medium according to the invention is a culture medium comprising the compounds of Table 1, preferably in the concentrations mentioned in Table 1, and also BSA, human insulin, human transferrin and optionally 2-mercaptoethanol, said medium also comprising a vitisin compound.

The vitisin compound, preferably vitisin-B, may be added to any one of the culture media described above, such as the amplification, differentiation and/or maturation media. In particular, the vitisin compound, preferably vitisin-B, may be present in the medium according to the invention at a concentration of between about 0.5 μM and about 1 mM, about 0.5 μM and about 500 μM, about 0.5 μM and about 200 μM, about 0.5 μM and about 100 μM, between about 0.5 μM and about 50 μM, between about 0.5 μM and about 40 μM, between about 0.5 μM and about 30 μM, between about 0.5 μM and about 20 μM, between about 0.5 μM and about 15 μM or between about 0.5 M and about 10 μM, between about 5 μM and about 1 mM, between about 5 μM and about 500 μM, between about 5 μM and about 200 μM, between about 5 μM and about 100 μM, between about 5 μM and about 75 μM, between about 5 μM and about 50 μM, between about 5 μM and about 40 μM, between about 5 μM and about 30 μM, between about 5 μM and about 20 μM, between about 5 μM and about 15 μM or between about 5 μM and about 10 μM, preferably between about 10 μM and about 1 mM, about 10 μM and about 500 μM, about 10 μM and about 200 μM, about 10 μM and about 100 μM, 10 μM and about 75 μM, between about 10 μM and about 50 μM, between about 10 μM and about 40 μM, between about 10 μM and about 30 μM, between about 10 μM and about 20 IM, preferably at a concentration of about 10 IM. Preferably, the vitisin compound is used at a concentration of between 1 μM and 500 μM, preferentially between 5 μM and 100 μM. In particular, the vitisin compound, preferably vitisin B, is added to the culture medium at a concentration of about 0.5, 1, 5, 10, 15, 20, 25, 30, 35, 40, 45, 50, 55, 60, 65, 70, 75, 80, 85, 80, 90, 95 or 100 μM.

The culture medium according to the invention comprising a vitisin compound may advantageously be used in processes for the preparation of hematopoietic stem cells (HSCs), multipotent progenitors (MPPs), common myeloid progenitors (CMPs), megakaryocyte-erythrocyte progenitors (MEPs), unipotent erythrocyte progenitors, erythrocyte precursors and red blood cells. In particular, the culture medium according to the invention comprising a vitisin compound may be used in any of the processes according to the invention, in particular as described above in the “Culture method” section.

The present invention thus also relates to the use of the cell culture medium according to the invention, in particular a culture medium suitable for the amplification, differentiation and/or maturation of hematopoietic cells and comprising a vitisin compound, in particular a medium as described in any one of the various embodiments above, for (i) the amplification and/or differentiation of hematopoietic stem cells (HSCs), multipotent progenitors (MPPs), common myeloid progenitors (CMPs), megakaryocyte-erythrocyte progenitors (MEPs) or unipotent erythrocyte progenitors and/or (ii) for the differentiation and/or maturation of erythrocyte precursors, and/or (iii) the production of unipotent erythrocyte progenitors and/or erythrocytes, notably via the processes according to the invention described below, and more particularly to stimulate the differentiation of HSCs, MPPs, CMPs and/or MEPs into unipotent erythrocyte progenitors; the differentiation of unipotent erythrocyte progenitors into erythrocyte precursors and/or to stimulate the maturation of erythrocyte precursors into erythrocytes.

The present invention also relates to the use of a vitisin compound to supplement a cell culture medium, in particular a cell culture medium suitable for the amplification and/or differentiation and/or maturation of hematopoietic cells, and more particularly a cell culture medium suitable for (i) the amplification and/or differentiation of hematopoietic stem cells (HSCs), multipotent progenitors (MPPs), common myeloid progenitors (CMPs), megakaryocyte-erythrocyte progenitors (MEPs) or unipotent erythrocyte progenitors and/or (ii) the maturation of erythrocyte precursors, and/or (iii) the production of unipotent erythrocyte progenitors and/or erythrocytes.

The present invention also relates to the use of a vitisin compound for (i) stimulating the amplification and/or differentiation of hematopoietic stem cells (HSCs), multipotent progenitors (MPPs), common myeloid progenitors (CMPs), megakaryocyte-erythrocyte progenitors (MEPs) or unipotent erythrocyte progenitors and/or (ii) stimulating the maturation of erythrocyte precursors, and/or (iii) stimulating the production of unipotent erythrocyte progenitors and/or erythrocytes. The vitisin compound can be simply added to a basic medium, preferably a basic medium suitable for the requirements of hematopoietic cells. The vitisin compound is preferably added to a culture medium chosen from an amplification medium, a differentiation medium and a maturation medium, notably as defined above. The vitisin compound may notably be added so as to be present in the supplemented medium at a concentration as defined above.

### Compositions Based on Vitisin Compounds and Use Thereof

According to another aspect, the present invention relates to the use of a vitisin compound for promoting or stimulating the production of hematopoietic cells, in particular erythrocytes, in an individual, in particular an individual suffering from hemopathy.

The present invention also relates to the vitisin compound as a medicament for the treatment of a hematological pathology. The term “medicament”, as used herein, encompasses drugs for human and animal use in human and veterinary medicine and refers to any pharmacologically acceptable substance that affords a therapeutic and/or beneficial effect.

The present invention also relates to a composition, in particular a pharmaceutical composition comprising a vitisin compound for the use thereof in the treatment of a hematological pathology or the treatment of patients who have suffered blood loss.

As used herein, a “pharmaceutical composition” denotes a preparation of an active agent with other optional chemical components such as physiologically appropriate supports and/or excipients. The pharmaceutical composition according to the invention encompasses pharmaceutical compositions used in human medicine and pharmaceutical compositions used in animal medicine, i.e. veterinary compositions.

The term “treatment” refers herein to the production of a desired pharmacological and/or physiological effect. The effect may be prophylactic in terms of total or partial prevention of a disease or symptom and/or may be therapeutic in terms of partial or complete cure of a disease and/or of an adverse effect attributable to the disease. The term “treatment” as used herein covers any treatment of a disease in a subject, in particular a human, to: reduce the incidence and/or risk of relapse of the disease during a symptom-free period; relieve or reduce a symptom of the disease; prevent the disease from occurring in a subject who may be predisposed to the disease but has not yet been diagnosed as having it; inhibit the disease, i.e. arrest its development (for example, reduce the rate of progression); reduce the frequency of episodes of the disease; and relieve the disease, i.e. cause a total or partial regression of the disease.

The terms “blood disease”, “hemopathy” or “hematological pathology” are interchangeable and refer herein to a disease affecting blood components (blood cells such as erythrocytes, leukocytes and platelets) or other components (hemoglobin, blood proteins) or affecting blood production or its mechanisms (coagulation, etc.). In particular, the hemopathy envisaged by the invention is a disease linked to damage to red blood cells or insufficient production of hematopoietic cells, preferably red blood cells.

The hematological disease may notably be selected from sickle cell disease, myeloproliferative syndromes (leukemias, Vaquez disease), aplastic anemia, and also anemias of other etiologies.
The pharmaceutical composition or vitisin compound may also be used for the treatment of insufficient production of hematopoietic cells, preferably red blood cells, caused by another disease, for example cancer.
The pharmaceutical composition or vitisin compound may also be used for the treatment of patients who have suffered severe blood loss, notably in the context of patients requiring blood transfusion.
In particular, transfusion occurs in patients with hemoglobin levels below 10. Blood transfusion consists in injecting blood or a blood component intravenously. The pharmaceutical composition or the vitisin compound may thus be used in addition to a blood transfusion.
The pharmaceutical composition according to the invention comprises a vitisin compound as active principle.
In one embodiment, the pharmaceutical composition may also contain at least one additional pharmaceutical active principle. The term “active pharmaceutical principle” means any compound or substance whose administration has a therapeutic effect or a beneficial effect on the health or general condition of a patient or subject to whom it is administered.

The formulation of such a pharmaceutical composition may vary as a function of the route of administration and dosage for which the composition is intended to be used.

The composition according to the invention may also comprise a pharmaceutically acceptable carrier. As used herein, the terms “pharmaceutically acceptable carrier” or “pharmaceutically acceptable excipient” or “pharmaceutically acceptable vehicle” are interchangeable and comprise any compound or combination of compounds which are known to those skilled in the art to be useful in the formulation of pharmaceutical or veterinary compositions. A person skilled in the art know how to select the most appropriate vehicles and excipients for the preparation of a given type of formulation.

The vitisin compound or pharmaceutical composition may be used for administration to a subject. The terms “individual”, “host”, “subject” and “patient”, are used herein interchangeably, and denote an animal, preferably a mammal, and more particularly a human. Preferably, the patient treated is a human being, for example a child or an adult. In particular, the subjects envisaged by the invention are affected by a hematological pathology or another pathology inducing insufficient production of hematopoietic cells, preferably red blood cells, for example cancer.

The present invention also relates to a method for treating a hematological pathology or other pathology inducing insufficient hematopoietic cell production. The treatment methods according to the invention generally involve administering to an individual in need thereof an effective amount of the vitisin compound or of a composition comprising a vitisin compound. In particular, the treatment is either for curative or preventive purposes, so as to limit or delay the onset of a hematological pathology or another pathology inducing insufficient hematopoietic cell production or a symptom associated with the disease.

The therapeutically effective or sufficient amount of a vitisin compound or of a pharmaceutical composition comprising said compound is an amount enabling the desired effect to be obtained, namely an effect promoting the production of hematopoietic cells, in particular red blood cells, by the patient.

In a particular embodiment, a conventional treatment known to those skilled in the art for treating a hematological pathology can be complemented by administration of a vitisin compound according to the invention. For example, administration of the vitisin compound may be combined with the administration of erythropoietin or a blood transfusion. Administration of the vitisin compound may be performed before, during and/or after the conventional treatment envisaged for the disease to be treated.

### Kit

In a final aspect, the present invention also relates to a kit comprising the compounds for the production of a cell culture medium according to the invention, in particular as described above in the “Culture medium” section, said medium not comprising any vitisin compound, said kit also comprising a vitisin compound.
In particular, the present invention relates to a kit comprising:
a cell culture medium, in particular a medium for the amplification, differentiation and/or maturation of hematopoietic cells, preferably as described above in the “Culture medium” section, said medium not comprising any vitisin compound; and
a vitisin compound, and
optionally, hematopoietic cells as described above in the “Erythropoiesis and hematopoietic cells” section, in particular HSCs, MPPs, CMPs, MEPs, unipotent erythrocyte progenitors and/or erythrocyte precursors; and
optionally, a guide containing instructions for the use of such a kit.
   Preferably, the kit comprises:
a cell culture medium, in particular as described above under the “Culture medium” section, preferably an IMDM medium and/or a Stem Span II medium, said medium not comprising any vitisin compound;
one or more compounds chosen from erythropoietin (EPO), Stem Cell Factor (SCF), hydrocortisone (HC), growth factors, interleukin 3 (IL3), interleukin 6 (IL6), interleukin 11 (IL11), transferrin, heparin, insulin, thrombopoietin, FMS-type tyrosine kinase 3 ligand (Flt3-L), granulocyte colony-stimulating factor (G-CSF), L-glutamine, inositol, an antibiotic such as penicillin and/or streptomycin, folic acid, mono-thioglycerol, iron nitrate, iron sulfate, human serum albumin, serum or plasma, or any combinations thereof, preferably chosen from erythropoietin (EPO), Stem Cell Factor (SCF), hydrocortisone (HC), growth factors, interleukin 3 (IL3), interleukin 6 (IL6), interleukin 11 (IL11), BIT9500, FMS-type tyrosine kinase 3 ligand (Flt3-L), granulocyte colony-stimulating factor (G-CSF), thrombopoietin (TPO), an antibiotic such as penicillin and/or streptomycin, or any combinations thereof;
a vitisin compound,
optionally, hematopoietic cells as described above in the “Erythropoiesis and hematopoietic cells” section, in particular HSCs, MPPs, CMPs, MEPs, unipotent erythrocyte progenitors and/or erythrocyte precursors; and
optionally, a guide containing instructions for the use of such a kit.

Alternatively, the kit according to the invention may comprise:
a cell culture medium as defined above in the “culture medium” section and comprising a vitisin compound; and
optionally, hematopoietic cells as described above in the “Erythropoiesis and hematopoietic cells” section, in particular HSCs, MPPs, CMPs, MEPs, unipotent erythrocyte progenitors and/or erythrocyte precursors; and
optionally, a guide containing instructions for the use of such a kit.
   In particular, the present kit may comprise any ingredients required to produce the culture medium in dehydrated form, in a common container or in separate containers.
   The kit according to the invention may also comprise various materials and reagents for use in accordance with the present invention in suitable containers and packaging materials, notably pipettes, tubes or bottles.
   The kit may also comprise any means for collecting a biological sample such as blood, for example a syringe.

The present invention also relates to the use of a kit according to the invention for producing erythrocyte progenitors, erythrocyte precursors and/or erythrocytes, notably according to the processes of the invention described above.

As used herein, the term “comprising” or “comprises” is used in reference to substances, compounds or processes which are essential to the invention, but which are open to the inclusion of nonspecific elements, whether essential or not. The use of “comprising” indicates inclusion rather than limitation.

The term “and/or” as used herein is to be considered as a specific description of each of the two specified features or components, with or without the other. For example, “A and/or B” should be considered as a specific disclosure of each of the following elements: (i) A, (ii) B and (iii) A and B, as if each were presented individually.

The term “about” as used herein in relation to all values (including the lower and upper limits of numerical ranges) means any value having an acceptable variation of up to ±10% (for example, ±0.5%, ±1%, ±1.5%, ±2%, ±2.5%, ±3%, ±3.5%, ±4%, ±4.5%, ±5%, ±5.5%, ±6%, ±6.5%, ±7%, ±7.5%, ±8%, ±8.5%, ±9%, ±9.5%). Using the term “about” at the start of a list of values modifies each of them (i.e. “about 1, 2 and 3” refers to about 1, about 2 and about 3). Also, when a list of values is described (for example about 50%, 60%, 70%, 80%, 85% or 86%), the list includes all its intermediate and fractional values (e.g. 54% or 85.4%).

All references cited in the present patent application, including journal articles or abstracts, published patent applications, issued patents or any other references, are fully incorporated herein by reference, including all results, tables, figures and text presented in said references.

Other features and advantages of the invention will emerge more clearly on reading the following examples, which are given as nonlimiting illustrations.

### DESCRIPTION OF THE FIGURES

FIG. 1: Effect of vitisin B on the ex vivo amplification of hematopoietic progenitors, notably progenitors with erythrocyte potential CD34⁺CD133^{neg} in an amplification procedure performed using peripheral blood CD34⁺ cells. The data represent the absolute number at the end of the protocol of each phenotypically identified cell subpopulation. The data represent the mean of four experiments.

FIG. 2: Comparison of the effects of vitisin B with other resveratrol derivatives/analogs on the ex vivo production of progenitors with erythrocyte potential CD34⁺CD133^{neg}. The data represent a comparison of the proportions (A) and absolute numbers (B) of CD34⁺CD133^{neg} progenitors at the end of the protocol as a function of the molecules tested. The data represent the mean of five experiments.

FIG. 3: Effect of vitisin B on erythrocyte differentiation, in a simplified erythrocyte differentiation procedure (ERYTHRO 1 PROTOCOL, detailed in the experimental section) from peripheral blood CD34⁺ cells. The data represent the level of CD71 membrane expression (mean fluorescence intensity, relative arbitrary values) (A) and the relative proportions of cells having acquired GPA expression at their membrane (B) as a function of the dose of vitisin B in the culture medium. The data represent the mean of six experiments. The vitisin B amounts indicated correspond to concentrations of 5, 10, 15, 20 and 30 μM, respectively.

FIG. 4: Effect of vitisin B on erythrocyte differentiation, in a simplified erythrocyte differentiation procedure (ERYTHRO 1 PROTOCOL, detailed in the experimental section) from SP, CMP and MEP subpopulations isolated from peripheral blood CD34⁺ cells. The data represent the evolution during culture of the relative proportions of cells expressing GPA and CD71 at their membrane (A) and also the evolution of the level of CD71 membrane expression (mean fluorescence intensity, relative arbitrary values) (B) as a function of the presence or absence of vitisin B (10 μM) in the culture medium. The data represent the mean of three experiments.

FIG. 5: Effect of vitisin B on the total amplification of placental blood CD34⁺ cells according to an optimized three-phase production process (ERYTHRO 2 PROTOCOL, detailed in the experimental section). The graph represents the absolute number of cells obtained and counted during culture in the absence or presence of vitisin B (10 μM). CTRL: Absence of vitisin B in culture/vitisin B; I: Presence of vitisin B during phase I/vitisin B; II: Presence of vitisin B during phases I and II/vitisin B; III: Presence of vitisin B during all three phases of the protocol. The data represent the mean of two experiments.

FIG. 6: Effect of vitisin B on the differentiation of placental and peripheral blood CD34⁺ cells into erythrocyte cells. Expression kinetics of glycophorin A (GPA) and transferrin receptor 1 (CD71) were analyzed during erythrocyte cell differentiation in the presence or absence of the vitisin B molecule (10 μM). Panels A and B: CD34⁺ cells from placental blood, the data represent the mean of two experiments. The histograms represent the evolution during culture of the relative proportions (panel A) and amounts (panel B) of cells of phenotype CD71^{neg}GPA⁺. Panel C: CD34⁺ cells from peripheral blood, the data represent the mean of five experiments. The histograms represent the evolution during culture of the amounts of cells of phenotype CD71^{neg}GPA⁺.

FIG. 7: Effect of vitisin B on the amplification of BFU-E and CFU-E erythrocyte progenitors from placental blood CD34⁺ cells. The number of primitive (BFU-E, Panel A) and mature (CFU-E, Panel B) erythrocyte progenitors was estimated in the context of a clonogenic assay performed at D4, D8 and D11 of erythrocyte culture. The data represent the mean of four experiments.

### EXAMPLES

### Preamble—Main Steps

### 1. First Step—Molecule Testing on Hematopoietic Cell Amplification

Vitisin B was tested in a hematopoietic cell amplification protocol and compared with seven other stilbene-type molecules.
CD34⁺ cells, derived from homeostatic peripheral blood, were used in this experiment.

A single cell culture protocol dedicated to the global amplification of hematopoietic progenitors was used. This is the “AMPLIFICATION PROTOCOL” described below in part B.1. of the Materials and Methods section.

The results obtained during this step made it possible to identify the molecule “Vitisin B” as a molecule that spontaneously promotes the amplification of a sub-population with erythrocyte potential.

### 2. Second Step in the Study—Investigating the Effect of Vitisin B on Erythrocyte Cell Production

Vitisin B was tested to define its ability to optimize the production of erythrocyte progenitors and red blood cells in culture systems dedicated to erythrocyte differentiation.
Various cell populations of interest were tested:
Total CD34⁺ cells.
Different CD34⁺ subpopulations: Side Population (SP), CMP (Common Myeloid Progenitor), MEP (Megakaryocytic and Erythroid progenitor), and BFU-E (Burst Forming Unit Erythroid).

All these different cell populations are physiologically organized according to the hierarchy SP->CMP->MEP->BFU-E->Erythroblast/Recticulocyte/Red Blood Cell on the one hand, and SP->CMP-->Other Blood Cells on the other. These different populations thus contribute to the production of mature blood cells.

The study of the effect of vitisin B is thus directed towards cell populations having the potential to produce red blood cells (CD34⁺, SPs, CMPs, MEPs and BFU-Es).

These cell populations of interest are derived from two different cell sources, homeostasis peripheral blood (SSBP) and/or placental blood (USP). The cell populations of interest studied therefore come from one of these two sources, or optionally from both.
In particular, CD34⁺ cells are derived from peripheral and/or placental blood, SP, CMP and MEP cells from peripheral blood, and BFU-E cells from placental blood.

Two different culture protocols allowing differentiation of the cell populations of interest into erythrocyte cells were used:
a simplified two-phase protocol (described in Materials and Methods, section B.2.b)
an optimized three-phase protocol (described in Materials and Methods, section B.2.c) Table 2 below summarizes the study organization.

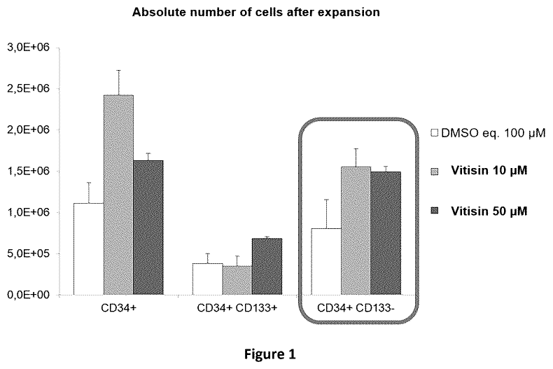

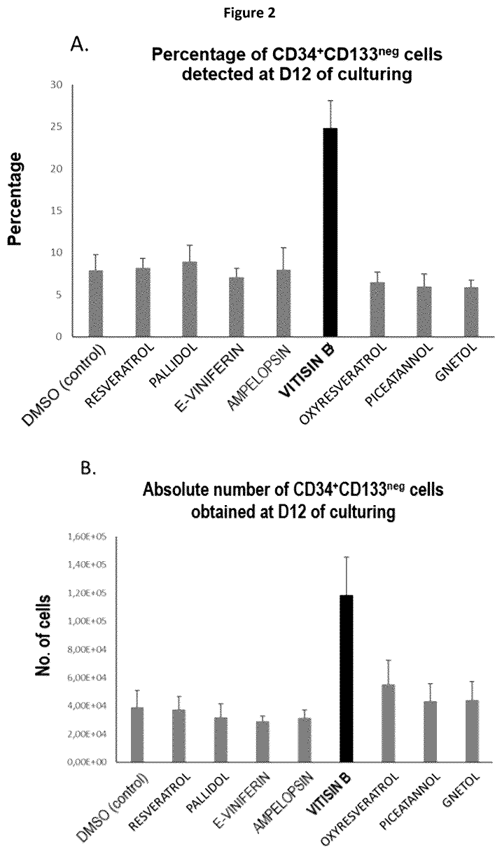

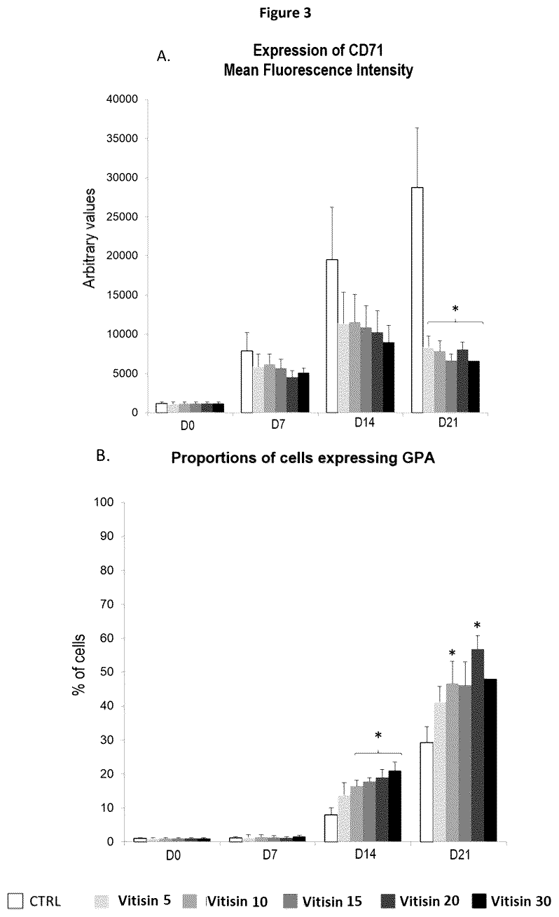

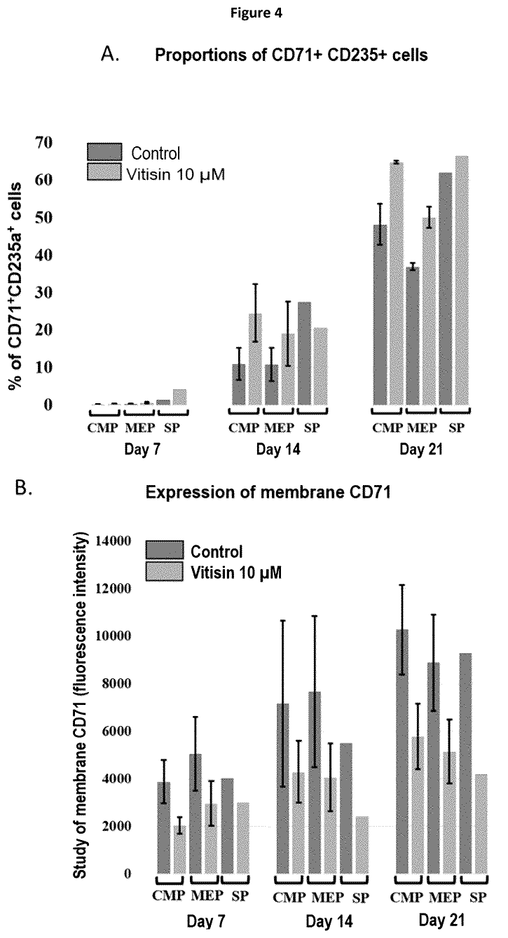

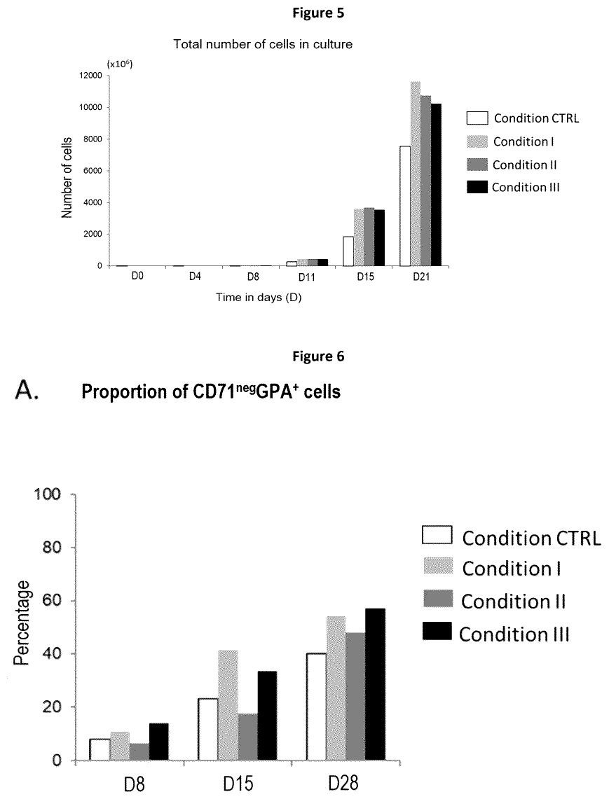

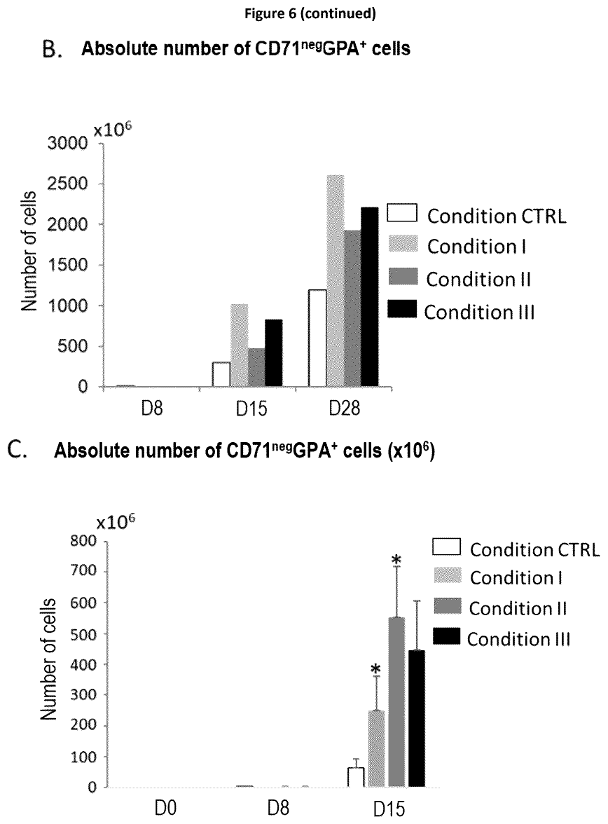

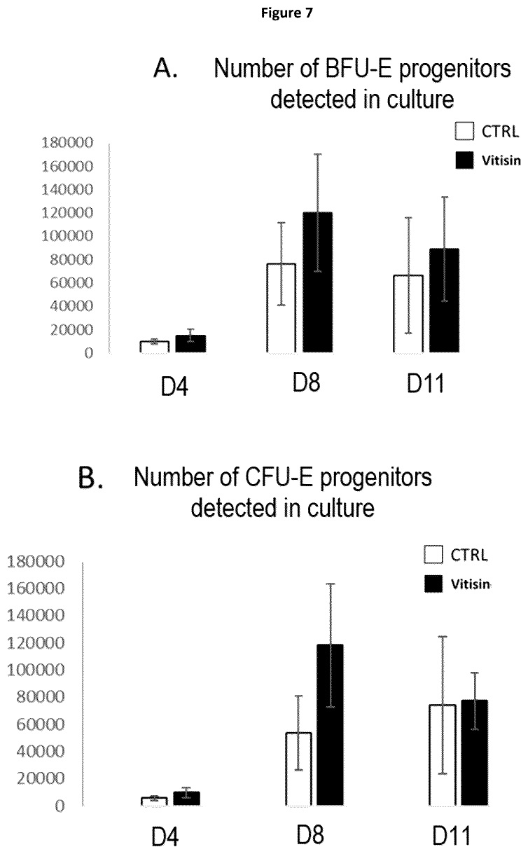

TABLE 2
Study organization.

| | Cell type | Source | Protocol | Molecules tested | Associated figure |
|---|---|---|---|---|---|
| Step 1 | CD34⁺ | peripheral blood | AMPLIFICATION | DMSO (control) | FIGS. 1 and 2 |
| | | | | RESVERATROL | |
| | | | | PALLIDOL | |
| | | | | E-VINIFERIN | |
| | | | | AMPELOSIN | |
| | | | | VITISIN B | |
| | | | | OXYRESVERATROL | |
| | | | | GNETOL | |
| Step 2 | CD34⁺ | peripheral blood | ERYTHRO 1 | VITISIN B | FIG. 3 |
| | CD34⁺ | placental blood | ERYTHRO 2 | VITISIN B | FIG. 6 |
| | SP | peripheral blood | PRESTIM + ERYTHRO 2 | VITISIN B | FIGS. 5 and 7 |
| | CMP | peripheral blood | ERYTHRO 1 | VITISIN B | FIG. 4 |
| | MEP | peripheral blood | ERYTHRO 1 | VITISIN B | FIG. 4 |
| | BFU-E | placental blood | Phase 1 ERYTHRO 2 | VITISIN B | FIG. 7 |
| | CFU-E | placental blood | Phase 1 ERYTHRO 2 | VITISIN B | FIG. 7 |

### I. MATERIALS AND METHODS

### A. Isolation of Populations of Interest

The phenotypes from which the different populations of interest are isolated from CD34⁺ cells (phenotype described in II.A.1 below) are summarized in Table 3 below with a cross-reference to the corresponding sections of “Materials and Methods”.

TABLE 3
Phenotypes used for the isolation of the different
CD34⁺ cell subpopulations used in the study.

| | | Materials & |
|---|---|---|
| | Phenotype | Methods section |
| SP | CD34⁺DCV^{low} | II.A.2. a |
| CMP | CD34⁺LIN^{neg}CD123⁺CD45RA^{neg} | II.A.2. b |
| MEP | CD34⁺LIN^{neg}CD123^{neg}CD45RA^{neg} | II.A.2. b |
| BFU-E | CD34⁺CD123^{neg}CD36^{neg}GPA^{neg}CD71^{low} | II.A.2.c |
| CFU-E | CD34^{neg}CD123^{neg}CD36⁺GPA^{neg}CD71^{high} | II.A.2.c |

### 1. Isolation of CD34⁺ Cells

This isolation is based on an immunomagnetic sorting technique. Selection of hematopoietic lineage cells having the CD34 surface antigen was performed by prior isolation of the mononuclear blood cell fraction from peripheral or placental blood leukocytes. Cells are isolated from blood by density gradient, using an isotonic Ficoll solution (d=1.077). The mononuclear cells are then cleared of residual blood platelets by density gradient (d=1.040), and the cell pellet is washed in a sucrose solution (d=1.0721) and then centrifuged (430×g, 10 min, 4° C.). After platelet removal, the pellets containing mononuclear cells are recovered and rinsed twice with selection buffer (PBS pH 7.2, EDTA 200 mM and human albumin (20%)) by centrifugation (430×g, 10 min, 4° C.). The pellet is then resuspended in 2 mL of selection buffer supplemented with DNase and Tegelin (0.5 μg/mL).

The CD34⁺ cells obtained were then isolated by a positive immunomagnetic selection procedure performed using a selection kit (Indirect kit CD34 microbeads kit, Miltenyi Biotec, Bergisch Gladbach, Germany) (Peytour et al., 2010).

### 2. Isolation of Different CD34⁺ Cell Subpopulations

a. Isolation of Side Population Cells (SP Cells)

SP cells are identifiable by virtue of their property of expelling certain dyes such as Hoescht or Dye Cycle Violet (DCV)). This expulsion takes place by means of calcium-dependent ATP Binding Cassette (ABCG2) pumps (Brunet de la Grange et al., 2013). To identify SP cells within previously isolated CD34⁺ cells, they are incubated with a DCV dye (5 μM) in DMEM⁺ medium (Dulbecco's Modified Eagle's Medium (DMEM), 2% FCS, 1% HEPES) for 2 h at 37° C. and in the dark. After incubation, all manipulations up to final cell sorting are performed on ice and in PBS⁺ medium (PBS, 2% FCS), a glucose- and calcium-depleted medium which stops the expulsion pumps. The cells are rinsed with cold PBS⁺ and centrifuged (430×g, 10 min, 4° C.). After resuspension of the pellet in PBS⁺, the cells are incubated with the viability marker 7AAD (7-aminoactinomycin-D) (1:100 000), allowing exclusion of any dead cells, and an anti-CD34-APC antibody (1:100 000) (Beckman Coulter). After incubation for 15 minutes, the cells are rinsed in PBS⁺, centrifuged (430×g, 10 min, 4° C.) and resuspended in PBS⁺. CD34⁺ cells having a low fluorescence intensity for the DCV marker (CD34⁺ SP cells) are analyzed (UV 350 nm or violet 405 nm), sorted and recovered using an ARIA cell sorter (Becton Dickinson).

b. Isolation of CMP and MEP Progenitors
The CD34⁺ cells previously isolated via an immunomagnetic technique were marked with anti-CD34-BV421 (1:100 000), anti-CD45RA-BB517 (1:100 000), anti-CD123-Alexa647 (1.5:100 000) antibodies (BD Pharmingen) and a cocktail of anti-LIN-PE antibodies (2:100 000) for 15 minutes. The LIN cocktail antibodies are directed against markers specific to mature cells (CD2, CD3, CD4, CD7, CD8, CD10, CD11b, CD14, CD19, CD20, CD56, CD235a). This LIN cocktail thus allows enrichment in “LIN^{neg}” immature cells by depletion of mature cells.
After incubation with these antibodies, the cells are rinsed with a solution of PBS⁺ (PBS+2% Fetal Calf Serum (FCS)) and centrifuged (430×g, 10 min. 4° C.).
After resuspension of the cells in PBS⁺, CMPs with the phenotype CD34⁺LIN^{neg}CD123⁺CD45RA^{neg} and MEPs with the phenotype CD34⁺LIN^{neg}CD123^{neg}CD45RA^{neg} are sorted and harvested using an ARIA cell sorter (Manz et al., 2002).
c. Isolation of BFU-E and CFU-E Progenitors
Placental blood CD34⁺ cells, previously isolated by immunomagnetic sorting, are marked with antibodies specific to surface antigens allowing them to target and discriminate BFU-E primitive erythrocyte progenitors, namely anti-CD34, anti-GPA, anti CD123 and anti-CD36 antibodies. Primitive erythrocyte progenitors (BFU-Es) are defined by the following phenotype: CD34⁺CD123^{neg}CD36^{neg}GPA^{neg}. Mature erythrocyte progenitors (CFU-E) are defined by the following phenotype: CD34^{neg}CD123^{neg}CD36⁺GPA^{neg}CD71^{high}.
To perform immunolabeling, the cells are incubated with a blocking solution for 10 minutes at 4° C., so as to saturate the Fc receptors, which are nonspecific antibody binding sites. Antibodies are added and the suspension incubated for 15 minutes in the dark at room temperature. The excess antibody is removed by washing the cells with 40 mL PBS/BSA buffer at 300×g for 10 minutes at 4° C., and the cell pellet is then resuspended at a concentration of 15×10⁶ cells/mL. This cell suspension is incubated for 10 minutes on ice with the fluorescent viability marker 7-AAD (7-aminoactinomycin D), which thus allows cell viability to be highlighted and dead cells to be excluded.
After phenotypic labeling, primitive erythrocyte progenitors (BFU-Es) are isolated via a cell sorting technique (ARIA BD).
After cell sorting of the population enriched in BFU-E primitive erythrocyte progenitors, it is washed in PBS buffer and centrifuged at 300×g for 10 minutes at 4° C. The cell pellet is resuspended.
Enrichment of the cell population with BFU-Es was verified by performing a CFC (Colony Forming Cells) clonogenic assay.

### B. Cell Culture Strategies

The cells were cultured under sterile conditions in a microbiological safety cabinet. Cells were grown in an incubator at 37° C. with a 5% CO₂ content.
Depending on the objective and the starting cell population, several culture strategies were used:

### 1. As Regards the 1^{st} Study Step: “AMPLIFICATION PROTOCOL”

This is a strategy for the amplification of hematopoietic progenitors. The culture is formed as follows: CD34⁺ cells from peripheral blood are isolated (as indicated in section II.A.1), and cultured for 12 days in Stem Span II medium (Stem Cell Technologies) supplemented with cytokines: SCF 100 ng/mL, FLT3-L 100 ng/mL, TPO 20 ng/mL, G-CSF 10 ng/mL. The amplification product is analyzed by quantification of proliferation (count on Malassez slides) and phenotypic analysis of CD34⁺ cell subpopulations by flow cytometry. Vitisin B is used at different concentrations, notably 10 and 50 μM. Vitisin B is also compared with the following stilbene-type molecules at 10 μM concentration: Resveratrol, Pallidol, E-Viniferin, Ampelopsin, Oxyresveratrol, Picateatannol, Gnetol

### 2. As Regards the 2^{nd} Study Step: “ERYTHRO 1 and ERYTHRO 2 PROTOCOLS”

This is a strategy for erythrocyte differentiation of the various hematopoietic cells of interest.
a. Amplification/Pre-Stimulation of SP Cells

This step relates to SP cells only. Due to the small number of SP cells that can be isolated (rare cells by nature) and also their very immature status, this sub-population is preferably amplified and pre-stimulated before being subjected to the erythrocyte differentiation protocol. Thus, the SP cells are pre-cultured in a liquid cell culture medium (Stem Span medium) supplemented with Granulocyte-Colony Stimulating Factor (G-SCF) (10 ng/mL), SCF (100 ng/μL), FLT3-L (100 ng/μL) and thrombopoietin (TPO) (100 ng/μL) for seven days. On conclusion of this step, the SP cells are subjected to an erythrocyte differentiation protocol.

b. ERYTHRO 1 PROTOCOL: Simplified Ex Vivo Erythrocyte Differentiation Protocol (in Two Phases)
This protocol was applied to CD34⁺ cells from peripheral and placental blood, pre-stimulated SP cells, CMP cells and MEP cells. For this culture, the cell populations of interest are isolated as indicated previously and cultured in IMDM culture medium supplemented as follows:
15% BIT 9500 (Stem Cell Technologies). BIT is a mixture of BSA (bovine serum albumin), insulin and transferrin.
Cytokines: IL3 at 10 ng/mL, IL6 at 10 ng/mL, SCF at 50 ng/mL.
Antibiotic mixture Penicillin—Streptomycin (100 U/mL) notably to avoid any bacterial contamination.
   The various cell populations were seeded in 24-well plates at 200 000 cells/mL for CD34⁺, CMPs and MEPs. All of the cells recovered after the “Amplification/pre-stimulation” step for SP cells (i.e. 5000-50 000 cells) were cultured in 24-well plates.
   This culture includes two phases:
Phase 1: D0 to D6, EPO-free culture
Phase 2: from D6, addition of 2 IU/ML EPO and extension of culturing up to D21 minimum and D28 maximum.

In order to test the effect of vitisin B in this context, the culturing is performed in the absence and presence of vitisin B:
CTRL condition: Absence of vitisin B throughout the entire protocol.
Test condition: Presence of vitisin B at 5, 10, 15, 20 or 30 μM for the entire duration of the protocol (phase 1+phase 2).
   During the culture protocol, the differentiation product is analyzed by quantifying proliferation (counting) and analyzing erythrocyte differentiation markers by means of flow cytometry, using the markers CD71 and CD235a.
   c. ERYTHRO 2 PROTOCOL: Three-Phase Erythrocyte Differentiation Protocol

This protocol was applied to CD34⁺ cells. Phase 1 of this protocol was in particular applied to BFU-E cells. This protocol was adapted from Vlaski et al. (Experimental Hematology 2009; 37:573-584).

The ex vivo erythrocyte production was established on a 28-day cell culture and involves three phases, namely 1) amplification of primitive erythrocyte progenitors, then 2) differentiation of said progenitors into erythrocyte precursors, and finally 3) the terminal stage of maturation with cell enucleation and development of mature erythrocytes.

i. Detailed Description of the Three Phases of the Protocol
Phase I of the protocol starts on DO with the seeding of 10⁴ cells/mL in 5 mL of supplemented IMDM medium as defined below (see section ii). On day 4, dilution of the culture is performed by recovering 3 mL, which is replaced with the same volume of fresh medium. On day 8, in order to allow these erythrocyte progenitors that are notably cytokine-dependent to survive and differentiate, cells from all conditions are resuspended at 3×10⁵/mL and incubated in 5 mL of IMDM medium in the presence of cytokines (human interleukin 3 (hIL3), Stem Cell Factor (SCF), hydrocortisone (HC) and erythropoietin (EPO)) for the following three days.
Phase II of the protocol starts on day 11, corresponding to the development of erythrocyte precursors. From day 11 to the end of the process, the cells are placed on a layer of mesenchymal stromal cells (MSCs) (coculture), allowing terminal maturation into functional enucleated cells by virtue of the contact between stromal cells and erythrocyte precursors.
   Suspended erythrocyte cells are seeded at a concentration of 3×10⁵/ml and co-cultured from day 11 to day 15 in fresh medium with cytokines (SCF, IL-3, EPO and HC) and 5% fetal calf serum (FCS) required for MSCs.
   On day 15, the suspended erythrocyte cells are removed from culture, washed, then reseeded onto a new adherent layer of MSCs and cultured for a further four days in 2 mL of fresh medium containing only EPO.
Phase III of the protocol begins on day 19. At this stage, the cells complete their maturation by the enucleation of orthochromatic erythroblasts to form reticulocytes, leading to the formation of erythrocytes. On day 19, the cells are washed and spread on a new layer of MSCs in 2 mL of fresh cytokine-free medium. On day 23, the cultures are fed with fresh medium until day 28, corresponding to the last day of culture in the erythropoiesis protocol.
   ii. Description of the Culture Medium

The cells are incubated in Iscove's Modified Dulbecco Medium (IMDM_PAN BIOTECH) supplemented with various reagents to obtain a medium promoting the growth of cells committed to the erythrocyte lineage, namely:
L-Glutamine (4 mM),
Streptomycin and penicillin (1%),
Inositol (40 μg/mL),
Folic acid (10 μg/mL),
Mono-thioglycerol (1.6×10⁻⁴ M),
Transferrin (120 μg/mL),
Insulin (10 μg/mL), N
Iron nitrate (90 ng/mL),
Iron sulfate (900 ng/mL)
Human serum albumin (10 mg/ml).

After preparation of the culture medium, cytokines are added extemporaneously to the culture medium:
Stem Cell Factor (SCF) at 100 ng/mL,
Interleukin-3 (IL-3) at 5 ng/mL,
Erythropoietin (EPO) at 3 IU/mL,
Hydrocortisone (HC) at 10⁻⁶ M.
   The CD34⁺ cells are incubated at 20% O₂ and 5% CO₂ at 37° C. for 28 days.
   iii. Description of the Culture Conditions Applied
   In order to test the effect of vitisin B in this context, various conditions were defined.

### For CD34⁺ Cells

CTRL condition: Absence of vitisin B throughout the entire protocol.
Condition I: Presence of vitisin B (10 μM) during phase I of the protocol.
Condition III: Presence of vitisin B (10 μM) during phases I and II of the protocol.
Condition III: Presence of vitisin B (10 μM) during the three phases of the protocol.

Analyses are performed on cells from each condition. The differentiation and maturation of erythrocyte cells are monitored by quantifying progenitors, analyzing the expression of GPA and CD71 markers, and also by morphological analysis.

### For BFU-Es

CTRL condition: cells are cultured for 7 days in the absence of vitisin B.
TEST condition: cells are cultured for 7 days in the presence of vitisin B at 10 μM.

### C. Strategies for Analyzing Cell Cultures

a. Amplification Analysis Strategies (STAGE 1 of the Study)

At the end of the hematopoietic cell amplification protocol, an analysis of cell proliferation and amplification of immature and committed cell populations is performed.

Cell proliferation is evaluated by counting cells on Malassez slides with Trypan blue exclusion of dead cells.

The amplification of cell populations is based on a phenotypic analysis relating to the evolution of the expression of membrane markers CD34 and CD133 by flow cytometry. These markers make it possible to define more or less immature cell populations organized hierarchically as follows: CD34⁺CD133⁺->CD34⁺CD133^{neg}->CD34^{neg}CD133^{neg}. To this end, cells are incubated with anti-CD34 (1:100,000) (BD Pharmingen) and anti-CD133 (1:100,000) (BD Pharmingen) antibodies for 15 minutes. The cells are then rinsed with a PBS solution and centrifuged (430×g, 10 min, 4° C.). After resuspension of the cell pellets in PBS, the cells are analyzed by flow cytometry (FACSCantoll, Becton Dickinson, San Jose, CA).

The proportions of each phenotypic population thus observed and the total amount of cells make it possible to calculate the rate of amplification of the most immature cells (CD34⁺CD133+) compared with the amplification of more committed progenitors (CD34⁺CD133^{neg} and CD34^{neg}CD133^{neg}).

b. Strategies for Analyzing Erythrocyte Differentiation (STAGE 2 of the Study)
i. Cell Counting and Phenotypic Analysis (Relates to ERYTHRO 1 and ERYTHRO 2 PROTOCOLS)

During and at the end of the various erythrocyte differentiation protocols, cell proliferation and cell differentiation are analyzed. Cell proliferation is evaluated by counting of cells on Malassez slides, with Trypan blue exclusion of dead cells. The different populations and culture conditions are also analyzed by flow cytometry, so as to observe the appearance of erythrocyte differentiation markers and thus cell differentiation kinetics. To this end, cells are incubated with antibodies specific for erythrocyte differentiation: anti-CD71 (1:100 000) (BD Pharmingen) and anti-CD235 (1:100 000) (BD Pharmingen) for 15 minutes. Labeling with an anti-CD34 antibody (1:100 000) (Beckman Coulter) is also performed in conjunction with the other antibodies, so as to monitor the loss of CD34 attesting to cell maturation. The cells are then rinsed with a PBS solution and centrifuged (430×g, 10 min, 4° C.). After resuspension of the cell pellets in PBS, the cells are analyzed by flow cytometry (FACSCantoll, Becton Dickinson, San Jose, CA).

The proportions of each phenotypic population thus observed and the total amount of cells make it possible to calculate the amounts of cells having reached the different stages of differentiation.

ii. Analysis of Erythrocyte Progenitor Production (Relates to ERYTHRO 2 PROTOCOL Only)

During the stages of differentiation into red blood cells, the populations of interest produce progenitors committed to the erythrocyte pathway, which can be demonstrated directly by their clonogenic capacities, i.e. their individual capacity to generate a colony of differentiated, morphologically identifiable cells during culture. To this end, the CFC (Colony Forming Cells) test is used. Committed progenitors are capable of generating colonies after culture in a semisolid medium composed of methylcellulose and supplemented with cytokines. Thus, committed progenitors (here BFU-Es and CFU-Es) produced during erythrocyte cultures can be counted.

This analysis of BFU-E and CFU-E production was performed only during phase 1 of PROTOCOL 2. Cells grown and harvested on days 4, 8 and 11 of phase 1 were seeded in a ready-to-use cytokine-supplemented methylcellulose H4034 kit (Stem Cell Technology, Meylan, France) at concentrations of 150 cells/250 L H4034 for day 4 and 300 cells/250

### II. RESULTS

### 1. Effect of Vitisin B and Various Antioxidant Molecules on the Amplification of Hematopoietic Progenitors (FIGS. 1 and 2).

In the context of hematopoietic progenitor amplification (AMPLIFICATION PROTOCOL), vitisin B induces an amplification of up to 250% of the total amount of progenitors of the CD34⁺ phenotype as early as the 10 μM dose (FIG. 1). It is also observed that vitisin B induces amplification of a particular subpopulation, CD34⁺CD133^{neg}, whose erythrocyte potential has already been shown by the inventors' team (Lapostolle et al. 2018 Hematologica Vol. 103 No. 10 (2018)). These CD34⁺CD133^{neg} progenitors with high erythrocyte potential consequently have the potential to produce mature red blood cells. Whether for the total CD34⁺ population or for the CD34⁺CD133^{neg} population, the effect of vitisin B already occurs at 10 μM, while increasing the dose to 50 μM induces no additional effect.

Comparison of vitisin B with other polyphenols (stilbenes) derived from resveratrol (RESVERATROL, PALLIDOL, E-VINIFERIN, AMPELOPSIN, OXYRESVERATROL, PICEATANNOL and GNETOL) at a dose of 10 μM shows that only vitisin B has a convincing effect on the proportion and total amount of CD34⁺CD133^{neg} cells (FIG. 2). This means that in the context of CD34⁺ cell amplification, vitisin B is capable of amplifying a subpopulation enriched in erythrocyte progenitors. This result in the context of CD34 amplification indicates a specific effect of vitisin B, where other well-known antioxidant molecules such as resveratrol have no effect.

Thus, only vitisin B allows the amplification of a population enriched in erythrocyte progenitors relative to the other molecules tested, demonstrating the singularity of vitisin B's effects.

### 2. Effect of Vitisin B on Erythrocyte Differentiation

a. In the Context of the ERYTHRO 1 Erythrocyte Differentiation Protocol
i. From Peripheral Blood CD34⁺ Cells (FIG. 3)
The effect of vitisin B was observed at the level of erythrocyte differentiation from peripheral blood CD34⁺ cells, evaluated according to a simplified protocol (ERYTHRO 1 PROTOCOL, presented in section B.2.b of Materials and Methods).
Under these conditions, the inventors observed that vitisin B induced faster and greater differentiation of CD34⁺ cells into erythrocyte precursors (FIG. 3). This effect is measured by flow cytometry on the basis of the expression of membrane antigens CD71 and CD235a (GlycoPhorin A, GPA) according to the differentiation sequence: CD71⁺GPA^{neg}->CD71⁺GPA⁺->CD71^{low}GPA⁺. The inventors observed an earlier acquisition of CD235a accompanied by a more rapid decline in CD71 expression (CD71^{low}), indicating differentiation.

On conclusion of these two protocols (AMPLIFICATION and ERYTHRO 1), two different and complementary effects of vitisin B are thus observed: an amplification of progenitors with erythrocyte potential and an acceleration of differentiation toward erythrocyte precursors.

ii. From SP, CMP and MEP Cells (FIG. 4)
Results identical to those described for peripheral blood CD34⁺ are found for the sorted CD34⁺ subpopulations: SPs, CMPs and MEPs. These results show an acceleration of erythrocyte differentiation in the presence of vitisin B (acquisition of the phenotype CD71⁺GPA⁺) for CMPs and MEPs (FIG. 4A), and also a reduction of up to 40% relative to the control in CD71 membrane expression for SPs, CMPs and MEPs as early as D7 in culture (FIG. 4B).
b. In the Context of the ERYTHRO 2 Three-Phase Erythrocyte Differentiation Protocol
i. Total Amplification of Placental Blood CD34⁺ Cells (FIG. 5)
The effect of the vitisin B molecule on the development of erythrocyte cells was also studied in a three-phase ex vivo erythrocyte production protocol according to an optimized protocol (ERYTHRO 2 PROTOCOL, presented in section B.2.c of Materials and Methods). FIG. 5 represents the number of erythrocyte cells in culture during the erythropoiesis protocol under the different conditions tested.
CTRL condition: Absence of vitisin B throughout the entire protocol.
Condition I: Presence of vitisin B during phase I of the protocol.
Condition II: Presence of vitisin B during phases I and II of the protocol.
Condition III: Presence of vitisin B during the three phases of the protocol.

In phase I (Days 0 to 11), cell proliferation was similar for each of the conditions tested. From the transition to phase II (Day 11), a sharp increase in cell proliferation was observed for the three conditions in which vitisin B was present, reaching a proliferation peak on day 21. In particular, condition I had 1.5 times more cells than the control condition (CTRL). These results are observed both using placental blood CD34⁺ cells and also using peripheral blood CD34⁺ cells in homeostasis.

Erythrocyte cell amplification was significantly increased in the presence of vitisin B in culture. Vitisin B stimulates the production of progenitor cells leading to the development of precursors and mature erythrocyte cells.

ii. Erythrocyte Differentiation Using CD34⁺ Cells from Peripheral Blood and Placental Blood (FIG. 6).
Erythrocyte differentiation was observed by expression of the CD71 (transferrin receptor 1) and GPA (glycophorin A) markers to confirm the effect of the vitisin B molecule on differentiation during the ex vivo erythropoiesis protocol (ERYTHRO 2 PROTOCOL).

The presence of the vitisin B molecule increases the percentage of cells with the phenotype CD71^{neg}GPA⁺ (FIG. 6A). Specifically, at day 8 of culture, less than 20% of cells express GPA. GPA expression is seen at day 15 and increases up to day 28 more rapidly in the presence of the vitisin B molecule (more than 50% of cells express it) compared with the CTRL condition (40%).

This effect is even more pronounced when it comes to the absolute number of cells with the phenotype CD71^{neg}GPA⁺ in culture (FIG. 6B). The presence of the vitisin B molecule in phase I of the protocol increases the number of cells with a differentiated phenotype CD71^{neg}GPA⁺. The same effect is observed with the absolute number of CD71^{neg}GPA⁺ cells produced from peripheral blood CD34⁺ cells (FIG. 6C).

These results show that the presence of the vitisin B molecule allows erythrocyte cells to differentiate more rapidly and efficiently, which ultimately reflects a more efficient production of red blood cells.

### 3. Effect of Vitisin B on BFU-E and CFU-E Erythrocyte Progenitors (FIG. 7)

The inventors also focused their attention on a population of particular progenitors, BFU-Es and CFU-Es. These progenitors are produced physiologically by differentiation of more immature cells, and also ex vivo when culture conditions allow. They are detected by methyl cellulose assays (see section C.b. ii in Materials and Methods), during which they produce colonies of mature erythrocyte cells (reticulocytes and erythrocytes). The inventors observed that the presence of vitisin B accelerated the appearance of primitive erythrocyte progenitors (BFU-Es; FIG. 7A) and also of more differentiated progenitors (CFU-Es; FIG. 7B) derived from BFU-Es.

These results show the advantage of vitisin B on a primitive hematopoietic cell population, thus improving the production of mature enucleated erythrocyte cells, i.e. reticulocytes and erythrocytes.

### III. CONCLUSION

The study of erythrocyte cells throughout this study made it possible to evaluate the effect of vitisin B on the amplification and differentiation of hematopoietic cells.

In particular, vitisin B exerts its stimulatory effect during the first developmental steps relating to the commitment of CD34⁺ cells to the erythrocyte lineage and the amplification of progenitors. It promotes the commitment of primitive CD34⁺ hematopoietic cells to erythrocyte progenitors, by increasing their amplification.

Vitisin B also allows accelerated differentiation of progenitors into erythrocyte precursors and their enucleation, leading to mature cells: reticulocytes and erythrocytes.

Placing hematopoietic cells in contact with vitisin B thus allows a higher yield of ex vivo red blood cells production, and speeds up the production process.
Specifically, these two effects combined together thus make it possible to overcome some of the bottlenecks observed in the production of red blood cells ex vivo, in particular low yields and production times.

1-19. (canceled)

20. An in vitro process for producing unipotent erythrocyte progenitors, comprising:
a) providing multipotent hematopoietic cells, preferably of human origin, selected from the group consisting of hematopoietic stem cells (HSCs), multipotent progenitors (MPPs), common myeloid progenitors (CMPs), megakaryocyte-erythrocyte progenitors (MEPs), and combinations thereof, and optionally amplifying said multipotent hematopoietic cells; and
b) inducing the differentiation of said multipotent hematopoietic cells into unipotent erythrocyte progenitors, and optionally amplifying said unipotent erythrocyte progenitors; and
c) optionally, recovering the unipotent erythrocyte progenitors obtained;
wherein the cells are placed in contact with a vitisin compound in step a) when it includes the cell amplification step and/or step b).

21. The method according to claim 20, said method further comprising:
d) inducing the maturation of the erythrocyte precursors into erythrocytes, and
e) optionally recovering the erythrocytes obtained;
wherein the cells are placed in contact with a vitisin compound in step a) when it includes the cell amplification step, step b), step c) and/or step d).

22. The process according to claim 20, wherein the cells are placed in contact with the vitisin compound in a culture medium that is suitable for the nutritional requirements of hematopoietic cells or that is suitable for the amplification and/or differentiation and/or maturation of hematopoietic cells.

23. The process according to claim 20, wherein the cells are placed in contact with the vitisin compound i) during the amplification and/or differentiation of multipotent hematopoietic cells or ii) during the amplification and/or differentiation of unipotent erythrocyte progenitors.

24. The process according to claim 21, wherein the cells are placed in contact with the vitisin compound during the amplification and/or differentiation of multipotent hematopoietic cells, and during the amplification and/or differentiation of unipotent erythrocyte progenitors and optionally during the maturation of erythrocyte precursors.

25. The process according to claim 20, wherein the cells are placed in contact with a vitisin compound for at least 1, 3, 5, 7, 10 or 12 days, during the amplification and/or differentiation of multipotent hematopoietic cells, and/or for at least 1, 3, 5, 7, 10 or 12 days, during the amplification and/or differentiation of unipotent erythrocyte progenitors.

26. The process according to claim 20, wherein the vitisin compound, when placed in contact with the cells, is present in the culture medium at a concentration of between 1 μM and 500 μM or between 5 μM and 100 μM.

27. The process according to claim 20, wherein the vitisin compound is vitisin B or a derivative thereof.

28. The process according to claim 27, wherein the vitisin B derivative is a vitisin B isomer or a vitisin B stereoisomer.

29. The process according to claim 20, wherein the vitisin compound is a vitisin derivative of formula:

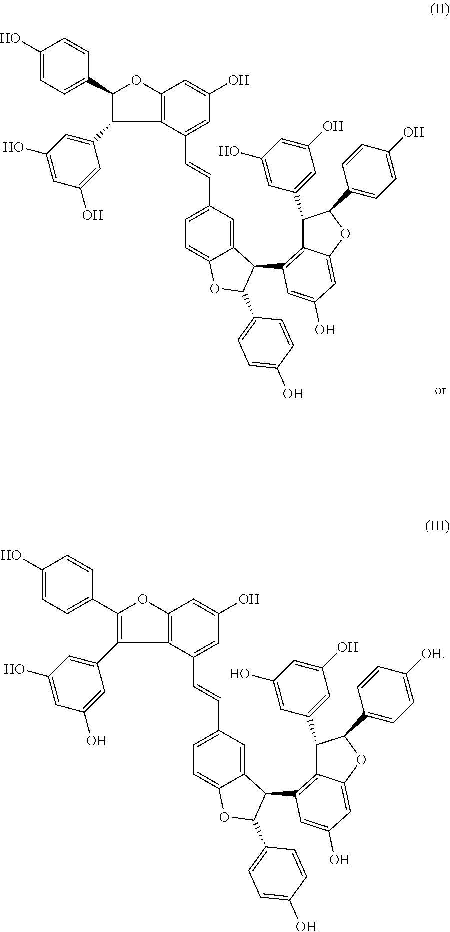

30. The process according to claim 20, wherein the vitisin compound is vitisin B.

31. An in vitro process for producing erythrocytes, comprising:
a) providing unipotent erythrocyte progenitors;
b) inducing the differentiation of said unipotent erythrocyte progenitors into unipotent erythrocyte precursors;
c) inducing the maturation of the erythrocyte precursors into erythrocytes; and
d) optionally recovering the erythrocytes obtained,
wherein the cells are placed in contact with a vitisin compound in step b) and/or step c).

32. A cell culture medium that is suitable for the amplification and/or differentiation and/or maturation of hematopoietic cells and comprising a vitisin compound.

33. The cell culture medium according to claim 32, selected from the group consisting of HSC, MPP, CMP, MEP cells, unipotent erythrocyte progenitors, erythrocyte precursors, and combinations thereof.

34. The cell culture medium according to claim 32, wherein the hematopoietic cells are HSCs or unipotent erythrocyte progenitors.

35. The cell culture medium according to claim 32, wherein the vitisin compound is present in the medium at a concentration of between 1 μM and 500 μM or between 5 μM and 100 μM.

36. The cell culture medium according to claim 32, wherein the vitisin compound is vitisin B or a derivative thereof.

37. The cell culture medium according to claim 36, wherein the vitisin B derivative is a vitisin B isomer or a vitisin B stereoisomer.

38. The cell culture medium according to claim 32, wherein the vitisin compound is a vitisin derivative of formula:

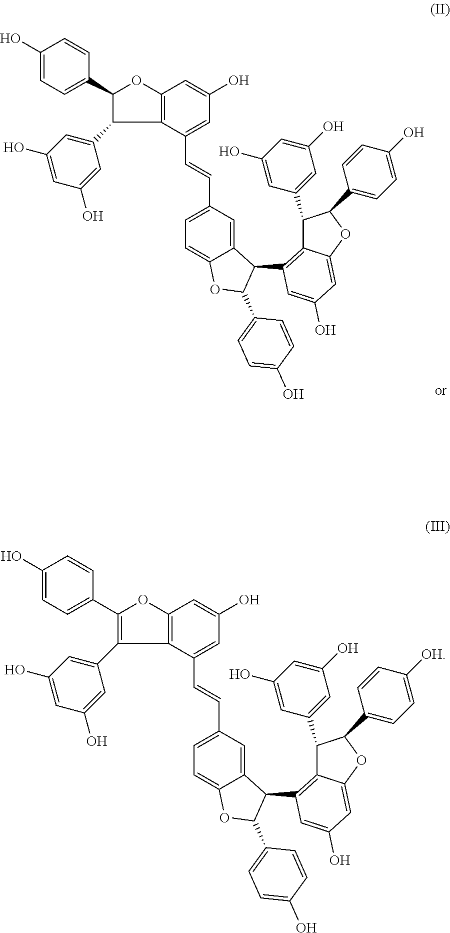

39. The cell culture medium according to claim 32, wherein the vitisin compound is vitisin B.
